(19) United States
(12) Patent Application Publication
(10) Pub. No.: US 2014/0328814 A1
(43) Pub. Date: Nov. 6, 2014

(54) ANTIMICROBIAL AND ANTIHYPERTENSIVE PROBIOTIC COMPOSITION, FOOD PRODUCT AND DIETARY SUPPLEMENT COMPRISING MICROORGANISM STRAIN LACTOBACILLUS PLANTARUM TENSIA DSM 21380 AND METHOD FOR SUPPRESSING CONTAMINATING MICROBES IN A FOOD PRODUCT

(71) Applicants: Epp Songisepp, Tartu (EE); Marika Mikelsaar, Tartu (EE); Merle Rätsep, Tartu (EE); Mihkel Zilmer, Tartu (EE); Pirje Hütt, Tartu (EE); Meeme Utt, Tartu (EE); Kristi Zilmer, Tartu (EE); Janne Uksti, Tartu (EE); Siiri Kõljalg, Tartu (EE)

(72) Inventors: Epp Songisepp, Tartu (EE); Marika Mikelsaar, Tartu (EE); Merle Rätsep, Tartu (EE); Mihkel Zilmer, Tartu (EE); Pirje Hütt, Tartu (EE); Meeme Utt, Tartu (EE); Kristi Zilmer, Tartu (EE); Janne Uksti, Tartu (EE); Siiri Kõljalg, Tartu (EE)

(73) Assignee: Oü Tervisliku Piima Biotehnoloogiate Arenduskeskus (Bio-Competence Centre of, Tartu (EE)

(21) Appl. No.: 14/303,272

(22) Filed: Jun. 12, 2014

Related U.S. Application Data
(63) Continuation-in-part of application No. 12/992,873, filed on Apr. 5, 2011, now abandoned.

(30) Foreign Application Priority Data
May 13, 2008 (EE) P200800026

(51) Int. Cl. A23L 1/30; A23L 3/3571; A61K 35/74

(52) U.S. Cl. CPC A23L 1/3014; A61K 35/747; A23L 3/3571
USPC 424/93.45; 426/61

(57) ABSTRACT

The present invention relates to an antimicrobial and antihypertensive probiotic compositions comprising microorganism strain Lactobacillus plantarum Tensia DSM 21380, e.g. food or pharmaceutical compositions or a dietary supplement compositions, food products (dairy products, e.g. fermented products or cheese). The invention also relates to method for suppressing pathogens and non-starter lactobacilli in food product and method for extension of shelf life of food product by the means of Lactobacillus plantarum Tensia DSM 21380.

### CROSS-REFERENCE TO RELATED APPLICATIONS

This application is a continuation-in-part and claims the benefit under 35 U.S.C. §120 of U.S. application Ser. No. 12/992,873, filed Nov. 15, 2010, the entire contents of which are incorporated herein by reference, which is a 371 National Stage of International Application No. PCT/EE2009/000005, filed May 12, 2009, the entire contents of which are incorporated herein by reference, which claims priority from prior EE Application No. P200800026, filed May 13, 2008.

### TECHNICAL FIELD

The present invention belongs to the field of biotechnology and is used in food industry and medicine. The invention relates to antimicrobial and antihypertensive probiotic microorganism strain Lactobacillus plantarum Tensia DSM 21380 and its use in the composition of a functional food e.g. cheese or dietary supplement and for preparation of antihypertensive medicine.

### BACKGROUND ART

Lactobacilli used for production of fermented foods have been consumed for centuries. During the past decades lactobacilli have widely been used as probiotics. Probiotics are live microorganisms of human origin, which, when administered in adequate amounts, confer a health benefit on the host. Commonly probiotics are used for creating functional foods. Food can be regarded as functional, if beyond adequate nutritional components it contains some natural additives (pre- or probiotics), which beneficially affect one or more target functions in the body, either improving the state of health and well-being and/or reducing disease risk.

Probiotic products may be conventional foods (yoghurt, quark, curds) or dietary supplements (freeze-dried microbial cultures)

In European countries several probiotics supplied with health claims are marketed

### Antimicrobial Probiotics

Antimicrobial probiotics against opportunistic pathogens and food-borne pathogens e.g. Salmonella have been described in patent applications. In WO2008/039531 (Little Columet Holdings KKC) the application of strains of Lactobacillus acidophilus, L. bulgaricus, L. casei, L. paracasei, L. fermentum, L. plantanum, L. rhamnosus, L. salivarius, Bifidobacterium bifida, B. infantis, B. animalis subsp. lactis, B. longum, Streptococcus thermophilus, Enterococcus faecalis and E. faecium is described. The oral administration of these strains helps to suppress Campylobacter jejuni, E. coli, S. aureus, Vibrio cholera, Bacteroides sp, Clostridium sp, Klebsiella sp, Listeria sp, Proteus sp, Salmonella sp, Shigella sp and Veillonella sp in gastrointestinal tract.

Patent application WO2008/016214 (Bioneer Corporation) describes a Lactobacillus gasseri strain BNR17 with antimicrobial activity against E. coli, S. aureus, S. typhimurium, B. cereus, L. monocytogenes and P. mirabilis.

Persons skilled in the art are aware of the problem caused in cheese by non starter lactobacilli (NSLAB), which appear during cheese ripening. These microbes can cause unwished proteolysis, appearance of various undesirable flavour compounds and loss of commercial quality of cheese.

Affecting the counts of NSLAB during cheese maturation process with pH regulators, antioxidants and preservatives (NaCl) has failed. The addition of different antimicrobial compounds producing (lactic acid and acetic acid, H₂O₂) antagonistic microbes incl. probiotic lactobacilli into cheese has also been ineffective. H₂O₂ producing probiotic lactobacilli have been described by several authors (Ouwehand, A. C. Westerlund, Antimicrobial components of lactic acid bacteria. In: Lactic acid bacteria: microbiological and functional aspects. Eds. Salminen, S; Wright, A., Ouwehand, A. C. 2004, pp 375-395, New York, Marcel Dekker; Hütt, P., Shchepetova, J., Lõivukene, K., Kullisaar, T., Mikelsaar, M. Antagonistic activity of probiotic lactobacilli and bifidobacteria against entero- and uropathogens. J. Appl. Microbiol., 2006, 100, 1324-1332).

Lactobacilli of human and plant origin adapt poorly in cheese environment rich in protein and fat and poor in carbohydrates, which is atypical for them. Therefore, only few probiotic lactobacilli comprising cheeses are available (Gardiner, G, Ross, R. P, Collins, J. H, Fitzgerald, G, and Stanton. C. Development of a probiotic cheddar cheese containing human derived Lactobacillus paracasei strains, Appl. Environ. Microbiol., 1998, 64, 6: 2192-2199; Ross, R P, Fitzgerald, G F, Collins, J K, O'sullivan, G C, Stanton, C G. Process for the manufacture of probiotic cheese, U.S. Pat. No. 6,872,411, Patent application WO 99/62348 AI), in which the added probiotic bacteria sustain their ability to multiply—a prerequisite for the expression of their physiological-biochemical properties. However, in cheese, that reportedly contained probiotic LGG, the presence of the strain was undetectable (Coeuret V Probiotic lactobacilli from feed or cheese origin: enumeration, identification, properties and specific use. Thesis de Doctoral de Univ. De Caen Basse Normandie, France 2004; Coeuret V, Dubernet S, Bernardeau M, Gueguen M, Vernon Jp. Isolation, characterization and identification of lactobacilli focusing mainly on cheeses and other dairy products. Lait, 2003, 269-306).

The incorporation of probiotic lactobacilli with strong antagonistic activity against pathogens into food product and into human organism helps to avoid food-borne infections. The ability of live lactobacilli (LB) to produce bacteriocins is an essential prerequisite for suppression and elimination of related gram-positive as well as gram-negative bacteria, including food-borne pathogens. Bacteriocins help to improve quality of a food product and prolong its shelf-life. Natural synthesis of aforementioned antimicrobial substances by LB in food product or application of synthetic analogues in food products is essential in the case of food products low production temperature and NaCl concentrations e.g. spread cheeses and in countries with warmer climate (Lindgren and Dobrogosz Antimicrobial compounds, including bacteriocins produced by LAB increase the self life of the product, FEMS Microbiol Rew 1990, 87 149-164).

L. plantarum and L. fermentum strains, isolated from boza have a property to produce bacteriocins that inhibit gram-positive microbes. These bacteriocins are either proteins or peptides with antimicrobial properties (Mollendorff J. W., Todorov, S. D., Dicks, L:. M:. T. Comparison of Bacteriocins Produced by Lactic-Acid Bacteria Isolated from Boza, a Cereal-Based Fermented Beverage from the Balkan Peninsula Current Microbiol. 2006, 53; 209-216). Production of plantaricins is typical to several L. plantarum strains. Sub-class IIa includes food-borne pathogens inhibiting bacteriocins, sub-class IIb harbours di-peptide bakteriocins of wide activity range: plantaricin EF and plantaricin JK, also plantaricin S (Maldonado A., Ruiz-Barba J L, Floriano B, Jimenez-Diaz R., Int J Food Microbiol. 2002, 77(1-2):117-124. “The locus responsible for production of plantaricin S, a class IIb bacteriocin, produced by L. plantarum LPCO10, is widely distributed among wild-type L. plantarum strains isolated from olive fermentation”). Well-known is the application of plantaricin S producing L. plantarum strains LP RJ1 and LPRJL2 in the vegetable fermentation process (WO02/05665, WO0060948, Consejo Superior de Investigaciones Cientificas). The latter belongs to similar group of antimicrobial compounds with colicins, which attack mostly members of the same species and genus (Heinaru A, Tallmeister E. Colicin susceptibility of shigellas and coli bacteria related to episomal resistance and colicinogenicity or dissociation into S-R forms. Geneetika, 1971, 7, 5, 113-122 (in Russian). Therefore they are promising substances for suppression of undesirable nonstarter lactobacilli.

Production of plantaricins is regulated with several genes, some of which occur occasionally, some regularly. Therefore the phenotypical differences in plantaricin production occur. Genomic locus, associated with bacteriocin production has been described in Lactobacillus plantarum strain 11. Two of these operons of genes where termed plnEFI and plnJKLR, each having gene pair plnEF and plnJK encoding two small cationic bacteriocin-like peptides with double-glycine-type leader sequences. (Diep, D. B., Havarstein, L. S. & Nes, I. F. Characterization of the locus responsible for the bacteriocin production in Lactobacillus plantarum C11. J Bacteriol 1996, 178, 4472-4483). No disclosure is available concerning L. plantarum strains that can suppress NSLAB and possess full set of plantaricin encoding genes EF and JK.

### Antihypertensive Probiotics

More people suffer from metabolic syndrome which symptoms include overweight, obesity, and raise of blood pressure, blood glucose content and several other risk markers of atherosclerosis. Foods rich in fat (incl. cheese) are not problem-free, as these foods can increase the risk of developing atherosclerosis, inflammation, type II diabetes and/or lipid peroxidation (Raff M., Tholstrup T., Basu S., Nonboe P., Sorensen Mont., Straarup E M. 138.509-514. A diet rich in conjugated linoleic acid and butter increases lipid peroxidation but does not affect atherosclerotic, inflammatory, or diabetic risk markers in healthy young men (American Society for Nutrition J. Nutr. 2008, 138:509-514).

Conjugated linoleic acid (CLA) refers to two naturally occurring isomers of 18-carbohydrate linoleic acid (LA, cis-9, cis-12-18:2). CLA forms in the process of natural biohydrogenisation and oxidation. CLA isomers are formed during biohydrogenation of linoleic acid in the rumen and also through conversion of vaccenic acid in the mammary gland. In optimal dosages the conjugated linoleic acid (CLA) isomers have high health amelioration potential: antimicrobial, antitumorigenic, antidiabetic, anti-obesity and anti-allergic. Antihypertensive effect has also been demonstrated by several research groups (Inoue K., Okada F., Ito R., Kato S., Sasaki S., Nakajima S., Uno A., Saijo Y., Sata F., Yoshimura Y., Kishi R. and Nakazawa H., Perfluorooctane sulfonate (PFOS) and related perfluorinated compounds in human maternal and cord blood samples: assessment of PFOS exposure in a susceptible population during pregnancy, Environ. Health Perspect. 112 (2004), pp. 1204-1207).

Supplementation of CLA in high amounts with food is, however, problematic. It has been demonstrated, that consumption of 5 g CLA daily leads to the increase of lipid peroxidation (LPO), expressed in the elevated concentration (83%) of 8-isoprostaglandine F₂ (Raff M., Tholstrup T., Basu S., Nonboe P., Sorensen Mont., Straarup E M. 138.509-514. A diet rich in conjugated linoleic acid and butter increases lipid peroxidation but does not affect atherosclerotic, inflammatory, or diabetic risk markers in healthy young men American Society for Nutrition J. Nutr. 2008, 138:509-514).

Best solution is the usage of lactobacillus strain possessing both the ability of moderate CLA production and physiologically relevant antioxidative capacity. Incorporation of physiologically relevant antioxidative LAB into food could control the increase of LPO and elevates the bio-suitability of fat-rich foods e.g. cheese. Lactobacillus fermentum ME-3 described in Tartu University patent EE04580 and in research articles possesses remarkable physiological antioxidative activity (Kullisaar T, Zilmer M, Mikelsaar M, Vihalemm T, Annuk H, Kairane C, Kilk A. Two antioxidative lactobacilli strains as promising probiotics. Int. J. Food Microbiol., 2002, 72, 215-224; Kullisaar, T., Songisepp, E., Mikelsaar, M., Zilmer, K., Vihalemm, T., Zilmer, M. Antioxidative probiotic fermented goats' milk decreases oxidative stress-mediated atherogenicity in human subjects. Br. J. Nutr., 2003, 90, 449-456; Truusalu, K. Naaber, P., Kullisaar, T., Tamm, H., Mikelsaar, R-H., Zilmer, K., Rehema, A., Zilmer, M., Mikelsaar, M. The influence of antibacterial and antioxidative probiotic lactobacilli on gut mucosa in a mouse model of Salmonella infection. Microbial Ecology in Health and Disease 2004, 16:4, 180-187).

Concentration of polyamines in human blood and exudation with urine fluctuate according to the consumption of food with different polyamines concentrations, but also in connection with endogenous polyamines producers in human intestinal microflora like E. coli and certain anaerobes (Marino M. Maifreni M, Moret S., Rondinini G. The capacity of Enterobacteriaceae sp. to produce biogenic amines in cheese. Letters of Microbiology, 2000, 31, 169-173). Several food products contain polyamines in relatively high concentrations e.g. oranges contain considerable amounts of putrescine 1330 μg/100 g (Larqué E., Sabater-Molina M., Zamora S. Biological significance of dietary polyamines. Nutrition, 2007, 23, 87-95.). Lactobacilli strains, which are able to produce moderate concentrations of CLA and polyamines and at the same time possess physiologically relevant antioxidativity have not been described.

Blood pressure is regulated by compounds like nitrogen monooxide (NO), lipid peroxidation (LPO), oxidized low density lipoproteins (ox-LDL) and components of glutathione red-ox system (GSSG/GSH), which modulate blood vessel stiffness and affect their vasoconstriction.

Moreover, the raise of LPO, ox-LDL, GSSG/GSH levels is one risk factor of atherosclerosis. Metabolism of acetylated spermidine provides additional possibilities for vasodilatory (blood pressure lowering) effect through acetylated form and polyamin ordinary forms (Myung C S, Blankenship J W, Meerdink D J. A mechanism of vasodilatory action of polyamines and acetylpolyamines: possible involvement of their Ca2+ antagonistic properties, J Pharm Pharmacol. 2000, 52:695-707).

Some peptides, harboured in milk possess blood pressure lowering ability through inhibition of angiotensine converting enzyme I (ACE) (Meisel, H. & Bocklemann, W. Bioactive peptides encrypted in milk proteins: proteolytic activation and tropho-functional properties. In: Proceedings of the sixth symposium on lactic acid bacteria: genetics, metabolism and applications. 19-23 September. Veldhoven (W. N. Konings, O. P. Kuipers, and J. Huis in't Veld., eds.) Kluver Academic Publishers, the Netherlands. 1999, pp. 207-215 (1999)).

Nitric monoxide NO belongs to bioactive compounds, having several beneficial effects like antimicrobial and anti-inflammatory activity and blood pressure lowering property, (Janeway, C A, Travers, P, Walport, M, Shlomchik, M J. 2005. Immunobiology: The Immune System in Health and Disease. New York, N.Y.: Garland Science Publishing). NO affects blood pressure though different pathways like relaxation of smooth muscles of blood vessels, ACE inhibition or promotion of endogenous protection mechanisms (preconditioning) (Jones S P, Bolli R The ubiquitous role of nitric oxide in cardioprotection. J Mol Cell Cardiol 2006; 40: 16-23).

Today only a few disclosures are available concerning probiotic microorganisms, which produce NO endogenously or promote NO endogenous production. Lactic acid bacteria with promote endogenous NO production are described in patent applications of Valio Ltd Nos EE00200230A and EE200200231. Patent application EE00200230A discloses lactobacillus strain L. helveticus LBK-16H, DSM 13137 with photolytic activity and which produces di- and tri-peptides. Those di- and tri-peptides derived form casein due to lactic fermentation are concentrated by nano-filtration and when applied into food products incl. fermented milk products, an antihypertensive peptides containing food has been created. Patent application EE200200231 discloses, that strain L. helveticus LBK-16H, DSM 13137 in addition to di- and tri-peptide production ability is also able to induce NO production in two cell lines (mice and human enterocytes). The strain assists blood pressure lowering through the production of peptides and NO. The authors note, that the bacterial cells were not the only NO producers (page 13, lines 8-9).

U.S. Pat. No. 7,183,108 (Compagnie Gervais Danone, 2007) discloses, that Lactobacillus casei possesses anti-inflammatory ability due to the increase of NO production (NO is produced by proinflammatory cytokine activated enterocytes), and vice versa—the bacterial NO production is down regulated when enterocytes are already activated by pro-inflammatory cytokines and bacterial lipopolysaccharides.

Korhonen et al. 2001 (Korhonen K, Reijonen T M, Remes K, Malmström K, Klaukka T, Korppi M. Reasons for and costs of hospitalization for paediatric asthma: A prospective 1-year follow-up in a population-based setting. Pediatr Allergy Immunol 2001:12:331-338) showed, that Lactobacillus rhamnosus GG could stimulate NO production by intestinal epithelial cells or by induction of proinflammatory cytocines. These authors pointed out the possible desirable connection of LGG and NO production.

European patent EP0951290 (Laboratories Standa S. A., 2002) discloses NO-producing propionibacteria applied into the composition for production of NO in gastrointestinal tract, presumably in enterocytes. Ibidem a strain of Lactobacillus farciminis is mentioned as a negative example (the producer of low NO amounts). U.S. Pat. No. 7,294,337 (Institut National de la Recherche Agronomique, 2007) however demonstrates that L. farciminis is able to produce NO in amounts sufficient for anti-inflammatory effect and suppression of pain after peritoneum stretch.

It has been disclosed that L. fermentum strain LF1 was able to produce NO aerobically in MRS broth without the presence of human cell culture. The L. plantarum strain DSM9843 (LP2), tested by same authors, was not able to act similarly, opposite to our strain L. plantarum Tensia DSM 21380 (J. Xu, W. Verstraete. Evaluation of nitric oxide production by lactobacilli. Appl. Microbiol. Biotechnol., 2001, 56:504-507). Incorporation into human organism a strain as a probiotic with aforementioned properties allows controlling the blood pressure due to the NO production ability of the strain and the property of NO to affect blood pressure pathways, as described above.

Besides the poor survival in food products, the selection of probiotic bacteria faces the problem of poor survival of strains thought the passage of the gastrointestinal environment. Therefore, such probiotic lactobacilli are needed, which are able to survive both in the upper as well as the lower parts of gastrointestinal tract. The international patent application WO 91/05850 (Tartu University et al., 1989) describes the strain L. plantarum 38, as beneficial for adjusting colonic microflora in the case of bacterial dysbiosis. European patent EP0554418 B1 (Probi AB, 1998) discloses the good colonization properties of the strain L. plantarum 299 (DSM 6595) in the human intestine and the application of the strain with oatmeal-drink for surgical patients against enteric infection. International patent application WO2007/003917 A1 (Matforsk A S et al, 2006) describes Lactobacillus plantarum DSM 16997 (DSM 17320) strains and a strain of L. pentosus (DSM17321) as starter microbes in meat products. According to authors, these strains can function as probiotics balancing human intestinal microflora through suppression of harmful bacteria and also commensial Escherichia coli. To prove these claims, the results of in vitro experiments are presented. However, no proof of pathogen suppression abilities in vivo (i.e. on animal model) is given. Besides, the suppression of non-starter lactobacilli (NSLAB) is not demonstrated, although survival of NSLAB in sausage environment is described at low pH values. Without proof the probiotic strains are attributed immunostimulatory and sepsis-lowering properties after surgical interventions and the improvement of patients' well-being through the administration of aforementioned probiotic strains.

Patent EE04097 B1 (Probi AB, 2003) discloses the application of Lactobacillus plantarum 299v (DSM 9843) for treatment of urinary infections, whereas the strain is able to adhere to the human colonic epithelium because of its mannose-specific adhesins and to compete with harmful urinary tract pathogens for adhesion sites. According to REA (restriction enzyme type) analysis all aforementioned strains differ from each other and from Lactobacillus plantarum Tensia DSM 21380 for the clearly distinct chromosome profile.

### DISCLOSURE OF THE INVENTION

The present invention relates to an antimicrobial and antihypertensive probiotic compositions comprising microorganism strain Lactobacillus plantarum Tensia DSM 21380 (e.g. food or pharmaceutical composition or a dietary supplement composition), food products (dairy products, e.g. fermented products or cheese). The composition can be used for production of antihypertensive medicine. The invention also relates to method for suppressing pathogens and non-starter lactobacilli in food product and method for extension of shelf life of food product by the means of Lactobacillus plantarum Tensia DSM 21380.

Lactobacillus plantarum Tensia DSM 21380 as antimicrobial probiotic produces antimicrobial substances—lactic acid, acetic acid, CLA, NO, H₂O₂ and possesses plantaricin II b class gene pairs EF and JK encoding antimicrobial compound. With its metabolites Lactobacillus plantarum DSM 21380 suppresses in vitro both non-starter lactobacilli and enteric pathogens. The strain is able to germinate and predominate among lactobacilli at low temperatures and in the condition of carbohydrate starvation (e.g. cheese).

In addition to antimicrobial activity Lactobacillus plantarum Tensia DSM 21380 is also an antihypertensive and antioxidative probiotic due to the production of NO, CLA and antioxidativity, produces in vitro small amounts of polyamines tyramine and putrescine and prevails in the intestinal microflora of volunteers after 3-week consumption of Lactobacillus plantarum Tensia DSM 21380 comprising cheese. The good colonization ability of Lactobacillus plantarum Tensia DSM 21380 in the human intestine was detectable in fecal samples both by DGGE profile and by methods of classical bacteriology. Lactobacillus plantarum Tensia DSM 21380 significantly activates the metabolism of putrescine and acetylated spermidine in the human body, accompanied by lowering both systolic and diastolic blood pressure.

### Description of the Strain

The microorganism strain Lactobacillus plantarum Tensia DSM 21380 was isolated from a faecal sample of a healthy child during a comparative study of the microflora of Estonian and Swedish children. The microorganism L plantarum Tensia DSM 21380 was isolated, by seeding the dilutions of the faeces of a healthy one-year old Estonian child (10⁻²-10⁻⁷ in phosphate buffer with 0.04% thioglycol acid; pH 7.2). The dilutions were seeded on freshly prepared MRS agar medium (Oxoid) and cultivated at 37° C. microaerobically. The strain was isolated according to the characteristic morphology of colonies and cell to Lactobacillus sp. A provisional and more precise identification followed, as described below.

The strain was isolated according to the characteristic morphology of colonies and cell to Lactobacillus sp. A provisional and more precise identification followed as described below.

The fact that the microbial strain Lactobacillus plantarum Tensia DSM 21380 originates from the intestinal tract of a healthy child, proves its GRAS (generally recognized as safe) status i.e. that this strain of microorganism is harmless for human organism and is suitable for oral application.

The microorganism Lactobacillus plantarum strain Tensia was deposited in Deutsche Sammlung von Mikroorganismen and Zellkulturen GmbH under the registration number DSM 21380 at 16 Apr. 2008.

Cultural-morphological characteristics were detected after cultivation on MRS agar and in MRS broth (OXOID). Cells of Lactobacillus plantarum Tensia DSM 21380 are Gram-positive, non-spore-forming rods of regular shape, occurring singly, in pairs or in short chains.

### Physiological-Biochemical Characteristics

The MRS broth was suitable for cultivation of the strain during 24-48 h in microaerobic environment, after which homogenous turbid growth occurred in the broth. Colonies on MRS agar plates after 48 h of growth at 37° C. in microaerobic conditions (atmosphere CO₂/O₂/N₂: 10/5/85) are round, 2-2.5 mm of diameter, sooth, entire, convex and white.

The optimal growth temperature is 37° C.; it multiplies also at 15° C. and 45° C. The optimal pH of the growth environment is 6.5.

Lactobacillus plantarum strain Tensia DSM 21380 is catalase and oxidase negative, facultatively heterofermentative, no gas is produced from glucose and no arginine hydrolysis.

Strain Lactobacillus plantarum Tensia DSM 21380 was identified on the basis of biochemical activity with API 50CHL System (bioMérieux, France) kit as Lactobacillus plantarum (Coincidence with the type strain: Excellent, ID %—99.9, T index—0.86).

The comparison with the reference strain Lactobacillus plantarum ATCC 14917 confirmed the preliminary identification by API 50CHL.

Carbohydrate utilization profile of Lactobacillus plantarum Tensia DSM 21380 according to API 50 CHL is as follows. Positive reaction for ribose, L-arabinose, galactose, D-glucose, D-fructose, D-mannose, mannitol, α methyl-D-mannoside, α methyl-D-glucoside, N acetyl-glucosamine, amygdalin, arbutine, esculine, salitsin, cellobiose, maltose, lactose, melibiose, D-arabinose, sachharose, trehalose, melezitose, D-raffinose, β-gentiobiose, D-turanose, gluconate, weak reaction for starch.

According to API ZYM test-kit (bioMérieux, France), Lactobacillus plantarum Tensia DSM 21380 possesses leucine arylamidase, acid phosphatase, α-glucosidase, β-glucosidase and acetoin activity. Weak reaction for valine arylamidase, naphthol-AS-BI-phosphohydrolase, β-galactosidase, cystine arylamidase, esterase (C4), esterase (C8), N-acetyl-β-glucosaminidase was detected.

The molecular identification of Lactobacillus plantarum Tensia DSM 21380 was confirmed by Internal-Transcribed Spacer Polymerase Chain Reaction (ITS-PCR in comparison with the reference strain Lactobacillus plantarum ATCC 14917 (FIG. 1).

Method. Strain identification was confirmed by (ITS-PCR) Internal-Transcribed Spacer Polymerase Chain Reaction in comparison with the reference strain Lactobacillus plantarum ATCC 14917.

The DNA extraction from Lactobacillus isolates was performed using lysozyme (Serva, Sweden; 20 mg/ml), mutanolysin (Sigma; 0.5 mg/ml) and proteinase K solutions (Fermentas, Lithuania; 14.6 mg/ml).

The DNA amplification was performed in 1×Taq polymerase buffer, containing 1.5U Taq polymerase (Fermentas, Lithuania), 0.5 μM of each primer (16S-1500F and 23S-32R; DNA Technology AS), 200 μM deoxynucleoside triphosphates (Amersham Pharmacia Biotech, Germany), 2 mM MgCl₂ and 2 μl of DNA under investigation.

Subsequently, the PCR product was restricted using a Taq I restriction enzyme (Fermentas, Lithuania). PCR products were separated by on a 2% agarose gel in 1×TBE buffer; voltage 100 V). The banding patterns were visualized in UV light and compared with reference stain L. plantarum ATCC 14917.

Lactobacillus plantarum Tensia DSM 21380 molecular fingerprints were compared with reference L. plantarum stain, using pulsefield-gel-electrophoresis (PFGE) (FIG. 2).

Method. For pulsfield-gel-electrophoresis (PFGE) procedure the lactobacillus strains were grown in MRS broth at 37° C. for 24 h. The cells were washed in SE buffer (75 mM NaCl, 25 mM EDTA, pH=7.4), density of the cell suspension was adjusted to 1.5 (OD₆₀₀). The DNA extraction from Lactobacillus isolates was performed in EC buffer (50 mM EDTA (pH 8.5), 0.5% Na-laurylsarcosine, 0.2% Na-deocycholate 2 mg/ml lysozyme, 10U mutanolysin), followed by 1 mg/ml proteinase K solution (100 mM EDTA-1% sarcosyl-0.2% deoxycholate, pH 8.0) containing buffer. Lysed probes were washed in TE buffer and cut to 2 mm, extracted overnight with enzyme 50U Not I (Bio-Rad). The electrophoresis was carried out in CHEF-DR II (Bio-Rad) for 22 h at 14° C. The banding patterns were visualized in UV light illuminator.

### Antibiotic Resistance

Method. Lactobacillus plantarum Tensia DSM 21380 was tested using antibiotic strips of the E-test (AB Biodisk, Solna). The MIC (minimal inhibitory concentration) was determined according to European Commission (EUC) suggested epidemiological break-points.

TABLE 1
Antibiotic resistance of Lactobacillus plantarum
Tensia DSM 21380

| | MIC* | MIC* | |
|---|---|---|---|
| | (μg/ml) | (μg/ml) | EUC antibiotic |
| | L. plantarum | Control strain | resistance MIC* |
| | Tensia | L. plantarum | breakpoint values |
| Antibiotic | DSM 21380 | DSM 21379 | (μg/ml) |
| Ampicillin | 0.25 | 0.19 | 4 |
| Gentamicin | 1.5 | 1 | 64 |
| Streptomycin | 16 | 6 | 64 |
| Erytromycin | 0.19 | 0.25 | 4 |
| Clindamycin | 0.032 | 0.016 | 2 |
| Tetracycline | 8 | 6 | 32 |
| Chloramphenicol | 2 | 2 | 8 |
| Cipro/ofloxacin | 32 | 32 | 4 |
| Quinopristin/ | 1 | 1 | 4 |
| dalfopristin | | | |

*minimal inhibitory concentration

Lactobacillus plantarum Tensia DSM 21380 did not harbour antibiotic resistance against most important antimicrobial preparations.

Somewhat higher MIC for ciprofloxacin was detected in normal range of wild strains described previously. Therefore, likely it is wild type stain (Vankerckhoven V, Huys G, Vancanneyt M, Vael C, Klare I, Romond M-B, Entenza J M, Moreillon P, D. Wind R, Knol J, Wiertz E, Pot B., Vaughan E. E, Kahlmeter G, Goossens H. Biosafety assessment of probiotics used for human consumption: recommendations from the EU-PROSAFE project. Trends in Food Science & Technology 2008; 19: 102e114) and no horizontal transfer of antibiotic resistance genes of Lactobacillus plantarum Tensia DSM 21380 during the application of the strain as a probiotic could be predicted.

### Functional Properties

### The Profile of Metabolites

Method. The profile of metabolites was determined by gas chromatographic method (Hewlett-Packard 6890) after incubation in microaerobic milieu for 24 h and 48 h (Table 2). L. plantarum strain was grown on MRS agar for 48 h microaerobically (10% CO₂). A suspension (McFarland 4.0 turbidity standard, 10⁹CFU/ml) of lactobacillus culture was prepared in 0.9% NaCl solution. 1.0 ml of this suspension was transferred to 9.0 ml of MRS broth. The metabolite concentration (mmol/l,) was detected using the capillary column HP-INNOWax (15 m×0.25 mm; 0.15 μm). The column temperature program was 60° C. 1 min, 20° C./min 120° C. 10 min; detector (FID) 350° C.

Electrochemical measurements of H₂O₂ were carried out with 24 h old intact cells in 500 μl of MRS broth with Apollo 4000 free radical analyzer (WPI, Berlin, Germany) and electrodes of type ISO-HPO2 and ISO-NOP.

ISO-HPO2 electrode signals were registered during 5-7 minutes. Mean signal strength was calculated. Each experimental point was measured in 4 independent parallels and each parallel was measured twice. H₂O₂ concentration was calculated according to the standard curves correlation with the strength of the electrodes signal.

TABLE 2
Acetic acid, lactic acid and succinic acid concentration
(mmol/l) of Lactobacillus plantarum Tensia DSM 21380
in MRS broth at microaerobic cultivation for 24 and 48 h and
H₂O₂ concentration (μg/ml) of intact cells

| | | | | | | | H₂O₂ |
|---|---|---|---|---|---|---|---|
| | Acetic acid | | Lactic acid | | Succinic acid | | (μg/ml) |
| | (mmol/l) | | (mmol/l) | | (mmol/l) | | intact cells |
| L. plantarum | 24 h | 48 h | 24 h | 48 h | 24 h | 48 h | 24 h |
| Tensia | 1.4 | 1.7 | 112.2 | 129.2 | 0.6 | 0.6 | 196.4 ± 128.8 |
| DSM 21380 | | | | | | | |
| DSM 21379 | 2.1 | 2.4 | 133.3 | 186.6 | 0.6 | 0.6 | 288.9 ± 175.8 |

Lactobacillus plantarum Tensia DSM 21380 produces substantial amounts of acetic acid, lactic acid and hydrogen peroxide. The concentration of the latter according to Apollo analyzer signals is however somewhat lower than that of the control strain.

### Antimicrobial Activity Against Pathogens and Non-Starter Lactobacilli

Lactobacillus plantarum Tensia DSM 21380 expresses in vitro on MRS agar medium antagonistic activity against non-starter lactobacilli and several enteric pathogens (Table 3). This property enables to use the strain for prolongation of shelf-life of food products.

TABLE 3
Lactobacillus plantarum Tensia DSM 21380 antimicrobial activity
against pathogens and non-starter lactobacilli on modified
MRS agar medium (pathogen growth inhibition zone, mm)

| Pathogen | Growth inhibition zone (mm) |
|---|---|
| Non-starter lactobacilli (NSLAB) | 8.6 ± 4.07 |
| Listeria monocytogenes ATCC51774 | 25.1 ± 1.7 |
| Yersinia enterocolitica | 13.5 ± 1.7 |
| Salmonella enteritidis | 25.2 ± 1.5 |
| S. enterica serovar Typhimurium | 22.8 ± 0.1 |
| Shigella sonnei | 25.1 ± 1.6 |
| Escherichia coli | 29.8 ± 3.7 |
| Enterobacter sakazakii | 18.5 ± 3.6 1 |
| Campylobacter jejuni | 12.9 ± 5.2 |

Lactobacillus plantarum Tensia DSM 21380 antimicrobial activity in vitro in streak-line procedure (antimicrobial effect of metabolites) was highest against E. coli, followed by growth inhibition of Salmonella sp., Shigella sp and Listeria sp. The lowest antimicrobial activity of Lactobacillus plantarum Tensia DSM 21380 was against other lactobacilli (NSLAB).

Antimicrobial Activity of Lactobacillus plantarum Tensia DSM 21380 at 4° C. Against Psychrophilic Pathogens

It is important to assess the antimicrobial activity of lactobacilli strains used as adjunct starters against psychrophilic food-borne pathogens at 4° C. (i.e. at food storage temperature).

Methods. Streak line procedure was used to assess the antimicrobial properties of the Lactobacillus sp (Hutt P, Shchepetova J, Loivukene K, Kullisaar T, Mikelsaar M. Antagonistic activity of probiotic lactobacilli and bifidobacteria against entero- and uropathogens. J Appl Microbiol. 2006; 100(6):1324-32).

The pathogens growth inhibition zone was measured in millimetres. Similarly to Hütt et al. (2006), the arithmetic mean and standard deviation were calculated according to the results of the used samples and on this basis, the antagonistic activity of strains was assessed (mm) as follows: at 4° C.—low <14.6; average 14.6-21.4; strong >21.4. All tests were repeated at least three times in parallel.

TABLE 3A
Antagonistic activity (mm) of L. plantarum Tensia (DSM 21380)
in streak line method at 4° C. incubation for 3 weeks

| Pathogen | L. plantarum Tensia (DSM 21380) |
|---|---|
| Listeria monocytogenes ATCC 13932 | 18.8 ± 1.0 |
| Listeria monocytogenes ATCC 51774 | 18.5 ± 3.0 |
| Y. enterocolitica (clinical strain) | 19.0 ± 2.9 |

Inhibition zone (mm): weak <14.6; average 14.6-21.4; strong >21.4

The metabolites of L. plantarum Tensia (DSM 21380) are able to inhibit the viability of psychrophilic L. monocytogenes and Y. enterocolitica at 4° C.

The effect of the growth environment and incubation temperature on the antimicrobial properties of L. plantarum Tensia DSM 21380

Methods. In order to evaluate the effect of the growth environment and temperature on the production of antimicrobial components by L. plantarum Tensia (DSM 21380), different incubation temperatures (15° C.; 30° C. and 37° C.) and different growth environments (MRS broth and standardised cheesemilk) were used. The lactobacillus was grown at incubation temperatures of 30° C. and 37° C. for 20-22 hours and at 15° C. for 30 days (cheese model).

Antagonistic Activity was Assessed with Ten-Day Intervals.

Experiments with the natural supernatant (pH range of 3.65 to 3.85) and the neutralized supernatant (pH range of 6.0±0.15) were carried out in parallel to exclude the effects of organic acids. Antimicrobial activity was assessed with agar drop diffusion test (Jimenez-Diaz R., Rios-Sanchez R. M., Desmazeaud M., Ruiz-Barba J. L. and Piard J.-C. (1993) Plantaricins S and T, Two New Bacteriocins Produced by Lactobacillus plantarum LPCO10 Isolated from a Green Olive Fermentation. Appl. Environ. Microbiol. 59, 1416-1424) Listeria monocytogenes ATCC 51774 ja ATCC 13932 suhtes.

TABLE 3B
Effect of different incubation temperatures (30° C. and 37° C.)
and pH of the supernatant (natural: 3.65 . . . 3.85 and neutralized:
pH 6.0 ± 0.15) of L. plantarum Tensia DSM 21380 grown in MRS on the
antagonistic activity of against Listeria monocytogenes
(pathogen growth inhibition in millimeters)

| | 30° C. | | 37° C. | |
|---|---|---|---|---|
| Strain | natural | neutral | natural | neutral |
| L. plantarum | 5.1 ± 1.4^{#} | 3.7 ± 2.6^{#} | 5.4 ± 0.5^{§} | 4.2 ± 2.0^{§} |
| Tensia (DSM 21380) | | | | |
| L. plantarum | 1.1 ± 2.1 | 1.1 ± 2.1 | 1.4 ± 2.2 | 1.4 ± 2.3 |
| DSM 21379 | | | | |

^{#}p = 0.03;
^{§}p = 0.04

The supernatant of L. plantarum Tensia (DSM 21380) grown in milk for 20-22 h at 30° C. and 37° C. had no antagonistic effect against tested L. monocytogenes strains.

The effect of bacteriocins, produced by lactobacilli is stronger in the acidic environment, as in this case all bacteria-produced antimicrobial compounds act synergistically. The optimum range of operation of bacteriocins is considered pH 5.0 . . . 7.0 (Atrih A., Rekhif N., Moir A. J. G., Lebrihi A. and Lefebvre G. (2001) Mode of action, purification and amino acid sequence of plantaricin C19, an anti-Listeria bacteriocin produced by Lactobacillus plantarum C19. Intern. Journal of Food Microbiol. 68, 93-104; Mezaini A., Chihib N.-E., Bouras A. D., Nedjar-Arroume, N. and Hornez, J. P. (2009) Antibacterial Activity of Some Lactic Acid Bacteria Isolated from an Algerian Dairy Product. J. of Environ. And Public Health. 1-6).

The antimicrobial activity of the supernatant of L. plantarum Tensia (DSM 21380) grown in MRS broth depended on the pH values—the antimicrobial activity of the natural supernatant (pH 3.65 . . . 3.85) against tested strains of Listeria sp was significantly better than that of the neutralised supernatant (Table 3B).

The antimicrobial activity of the supernatant of L. plantarum Tensia (DSM 21380) grown in MRS broth depended on the incubation temperature, being stronger at 37° C. (Table 3B).

TABLE 3C
The effect of low incubation temperatire (15° C.) and different growth environments
(MRS and milk) on the antimicrobial activity against Listeria sp within 30 days
of incubation in MRS broth and milk (pathogen growth inhibition in millimetres)

| | MRS | | | milk | | |
|---|---|---|---|---|---|---|
| Strain | Day 10 | Day 20 | Day 30 | Day 10 | Day 20 | Day 30 |
| L. plantarum | 5.6 ± 0.8 | 5.8 ± 1.3 | 7.1 ± 1.4 | 0 | 3.0 ± 2.3 | 6.0 ± 0.7 |
| Tensia | | | | | | |
| L. plantarum | 0 | 0.6 ± 1.1 | 1.9 ± 2.6 | 0 | 2.2 ± 2.8 | 0 |
| DSM 21379 | | | | | | |

The supernatant of L. plantarum Tensia (DSM 21380) grown at 15° C. in MRS broth had antagonistic activity from the first measurement (10^{th} incubation day). The anti-microbial properties against Listeria sp of the L. plantarum Tensia (DSM 21380) supernatant, separated from the milk was observed from the 20^{th} incubation day, and the antimicrobial properties of the supernatant retained and strengthened to some extent towards the end of the incubation period. The antimicrobial properties of the strains supernatant separated from the milk increased were significantly weaker (p<0.01) in comparison to the supernatant of lactobacilligrown in MRS broth.

Lactobacillus plantarum Tensia DSM 21380 Bacteriocin Genes

Method. Detection of Lactobacillus plantarum Tensia DSM 21380 bacteriocin encoding genes based on the Lactobacillus plantarum WCFS1 genes plnE, plnF plnJ and plnK. These genes encode bacteriocin precursor peptides (Lactobacillus plantarum WCFS1 genome annotation—Kleerebezem et al 2003. Complete genome sequence of Lactobacillus plantarum WCFS1. Proc Natl Acad Sci USA. 2003 Feb. 18; 100 (4): 1990-5). The PCR primers were designed using Primer Express®. Lactobacillus plantarum WCFS1 annotated genome was downloaded from NCBI genome browser (http://www.ncbi.nlm.nih.gov/Genomes).

The designed primers were designated as E1F, E1R, F1F, F1R, J1F, J1R, K2F, K2R (look: Sequence listing) and there use in PCR-reaction gave positive signals for all aforementioned sequences. L. plantarum BAA-793 NCIMB 8826 was used as positive control and strain L. plantarum DSM 21379 as negative control.

TABLE 4
Presence of amplified PCR products of genomic DNA
bacteriocin encoding genes plnE, plnF, plnJ and plnK
of Lactobacillus plantarum Tensia DSM 21380

| | Genes | | | |
|---|---|---|---|---|
| | plnE | plnF | plnJ | plnK |
| L. plantarum BAA-793 (NCIMB 8826) | + | + | + | + |
| L. plantarumTensia DSM 21380 | + | + | + | + |
| L. plantarum DSM 21379 | + | + | − | − |

The antagonistic activity of L. plantarum Tensia DSM 21380 among other antimicrobial compounds is also based on aforementioned gene products e.g. antimicrobial peptides. Peptides E & F and J & K should be expressed simultaneously to get max response via di-peptides.

Production of conjugated linoleic acid (CLA) by L. plantarum Tensia DSM 21380 Method. Production of CLA was determined in MRS broth (de Mann-Rogosa-Sharpe, Oxoid, UK) and in skim milk by spectrophotometrical measurements.

| | | |
|---|---|---|
| MRS broth | 39.9 | mg/l (moderate) |
| Skim milk | 19.2 | mg/l (moderate) |
| Cheese | 3.0 ± 0.3 | mg/g |

Production of nitrogen mono-oxide (NO) by L. plantarum Tensia DSM 21380 Method 1. Nitrogen mono-oxide production measurements were carried out with 24 h old intact cells in 500μ of MRS broth with Apollo 4000 free radical analyzer (WPI, Berlin, Germany) and electrodes of type. ISO-NOP electrode signals were registered during 5-7 minutes. Mean signal strength was calculated. Each experimental point was measured in 4 independent parallels and each parallel was measured twice. NO concentration was calculated according to the standard curves correlation with the strength of the electrodes signal.

TABLE 5A
NO concentration (μM) produced
by L. plantarum Tensia DSM 21380

| Strain number | NO concentrations (μM) |
|---|---|
| L. plantarum Tensia DSM 21380 | 2.6 ± 0.8 |
| L. plantarum DSM 21379 | 2.7 ± 1.2 |
| L. coprophilus | 2.1 ± 1.1 |
| L. plantarum | 2.1 ± 0.9 |
| L. paracasei ssp paracasei strain no 1 | 1.3 ± 0.8 |
| L. paracasei ssp paracasei strain no 2 | 1.8 ± 0.9 |
| L. paracasei ssp paracasei strain no 3 | 2.8 ± 1.6 |
| L. buchneri | 2.0 ± 1.1 |

L. plantarum Tensia DSM 21380 was one of the best NO producers in comparison with 10 other Lactobacillus sp strains.

Method 2. Nitric oxide production was measured in 24 h old microbial cells, incubated in 10 ml of MRS broth containing 3 or 30 mg of NaNO₃

The measurements were carried put with Apollo 4000 free radical analyzer as described above.

TABLE 5B
Production of NO (μM) by L. plantarum
Tensia DSM 21380 in vitro.

| | Concentration of | Concentration of NO (μM) |
|---|---|---|
| Strain | NaNO₃ | in MRS broth |
| | MRS broth | 0.0 ± 0.0 |
| L. plantarum | MRS broth | −0.1 ± 0.0 |
| Tensia DSM 21380 | 3 mg NaNO₃ | 4.5 ± 0.9 |
| | 30 mg NaNO₃ | 11.0 ± 2.2 |
| L. plantarum | MRS broth | 0.7 ± 0.4 |
| DSM 21379 | 3 mg NaNO₃ | 4.1 ± 5.3 |
| | 30 mg NaNO₃ | 5.4 ± 6.0 |

The amount of NO produced by L. plantarum Tensia DSM21380 is dependent on the NaNO₃ concentration in the medium.

The authorized concentration of NaNO₃ in salted or preserved products is 300 mg/kg and of cheese 150 mg/kg (http://www.riigiteataja.ee/ert/act.jsp?id=12937247).

Antioxidative Activity of Lactobacillus plantarum Tensia DSM 21380

Method. For the detection of TAA and TAS of the microbial cells, the strain L. plantarum Tensia DSM 21380 was incubated in MRS broth (Oxoid, U.K.) for 24 h at 37° C.

Microbial cells were harvested by centrifugation (1500 RPM, during 10 min) at 4° C. and the pellet was washed with isotonic saline (4° C.) and suspended in 1.15% KCl (Sigma, USA). The density of the suspension was OD₆₀₀ of 1.1×10⁹ bacterial cells ml⁻¹). Total antioxidative activity (TAA) was assessed by using the linolenic acid test (LA-test). (Kullisaar, T, Songisepp, Mikelsaar M, Zilmer, K, Vihalemm, T, Zilmer, M. British J of Nutrition. Antioxidant probiotic fermented milk decreases oxidative stress-mediated atherogenicity in human. 2003, 90, 2, 449-456) and total antioxidative status (TAS) was measured by commercial kit (TAS, Randox Laboratories Ltd., UK) (Table 6).

TABLE 6
Total antioxidative activity (TAA) and total antioxidative
status of (TAS) Lactobacillus plantarum Tensia DSM 21380

| Strain | TAA (%) | TAS (mmol/l) |
|---|---|---|
| L. plantarum Tensia DSM 21380 | 22 ± 5 | 0.05 ± 0.02 |
| L. plantarum DSM 21379 | 26 ± 1.2 | 0.13 ± 0.04 |

The values of TAA and TAS of the strain L. plantarum Tensia DSM 21380 were somewhat lower than of the strain L. plantarum DSM 21379.

L. plantarum Tensia DSM 21380 parameters were lower in comparison with the values of the antioxidative strain L. fermentum ME-3 (DSM14241) (ME-3, TAA: 29±0.7; TAS: 0.16±0.03).

In vitro polyamines production of Lactobacillus plantarum Tensia DSM 21380 Method. Microbial strains were suspended in physiological saline according to McFarland turbidity standard (10⁹ CFU/ml) and 0.5 ml of each strain suspension was seeded into decarboxylation medium (á 4.5 ml) and incubated at 37° C. for 4 days (Bover-Cid et al., 1999).

For detection of BA 200 μl of medium was derivatized for GC analyze by modified method of Nakovichi (Nakovich, L. Analysis of biogenic amines by GC/FID and GC/MS. Thesis, Virginia polytechnic institute, USA. 2003).

GC analyses were carried out by gas chromatograph HP 6890 Series GC System, with capillary colonna HP-5 19091J-413 (30 m×0.32 mm; 0.25 μm). The column temperature program 160° C. 1 min, 20° C./min 280° C. 15 min; and detector (FID) 300° C.

TABLE 7
Concentration of polyamines in vitro in decarboxylation medium Arena, M. E., Manca de Nadra, M.
C. Biogenic amine production by Lactobacillus. J. Applied Microbiology 2001, 90, 158-162.

| | Polyamines (μg/ml) and biogenic amines | | | | | | | | |
|---|---|---|---|---|---|---|---|---|---|
| | | | | | | | | | Histi- |
| | Arginine | | Glutamine | | Lysine | | Ornithine | | [symbol] |
| Sample | Putrescine | Cadaverine | Putrescine | Cadaverine | Putrescine | Cadaverine | Putrescine | Cadaverine | Histamine |
| L. plantarum | 0 | 0 | 0 | 0 | 0 | 0.3 | 0.5 | 0.6 | 0 |
| Tensia DSM | | | | | | | | | |
| 21380 | | | | | | | | | |
| L. plantarum | 0 | 0.4 | 1.2 | 0.5 | 0 | 0.4 | 1.9 | 0 | 0 |
| DSM 21379 | | | | | | | | | |
| E. coli ATCC | 18.4 | 1.7 | 12.3 | 18.4 | 1.8 | 240 | 1599.3 | 3.5 | 105.1 |
| 700336 | | | | | | | | | |

[symbol]

indicates data missing or illegible when filed

L. plantarum Tensia DSM 21380 produces small amounts of polyamine putrescine from ornithine. From biogenic amines, traces of cadaverine were detected. No production of histamine.

Angiotensin Converting Enzyme (ACE) Inhibitory Activity of L. plantarum Tensia DSM 21380 Fermented Milk

L. plantarum Tensia DSM 21380 was inoculated (2%, vol/vol) to 50 ml of pasteurized cheese milk. Inoculation was carried out under sterile conditions and the milk was kept for fermentation at 37° C. for 24 h. The experiment was repeated twice in two parallels. For the determination of the ACE inhibitory activity of the milk during fermentation, the whey fraction was used. The whey fraction was obtained as follows. The pH of milk was adjusted to 3.4 by addition of 50% lactic acid. The milk acidified with lactic acid and the milk fermented by L. plantarum TENSIA were centrifuged at 6000×g for 10 min; 10N NaOH was added to the supernatants to raise the pH to 8.3, and then the supernatant was centrifuged at 6000×g for 10 min.

The milk hydrolysates were centrifuged at 6000×g for 10 min. The supernatant was ultrafiltered through a 10 kDa cut-off filter (Millipore, USA) and in second step of fractionation through a 3 kDa cut-off filter by centrifugation (4000×g for 40 min at 15° C.). The final supernatant was used for the study (Praveesh B. V, Angayarkanni J., Palaniswamy M. Antihypertensive and anticancer effect of cow milk fermented by Lactobacillus plantarum and Lactobacillus casei. International Journal of Pharmacy and Pharmaceutical Science, 2011, 3, 5, 452-456).

The molar concentration of peptides in the supernatant was determined by quantitative ninhydrin (Sigma-Aldrich, USA) assay, a rapid and sensitive method for the quantitative determination of free amino groups. The technique involves the reaction of the free amine with ninhydrin under carefully controlled conditions and the determination of the resulting chromophore concentration in solution at 570 nm. Leucine (Sigma, USA) was used as standard for creating a standard curve.

The K-Assay® ACE Inhibition Screening Kit (Kamiya Biomedical Company, Seattle, USA) was used for measurement of ACE inhibitory activity. 3-Hydroxybutyryl-Gly-Gly-Gly (3HB-GGG) is utilized as a substrate for ACE, and the amount of cleaved 3-hydroxybutyric acid (3HB) from 3HB-GGG is measured by the enzymatic method. The inhibition activity was calculated using the following equation: ACE inhibitory activity (%)=[(A_{blank1}−Aₛₐₘₚₗₑ)/(A_{blank1}−A_{blank2})]×100, where blank1 is positive control (without ACE inhibition) and blank2 is reagent blank. Each sample was assayed in triplicate. For determination of IC₅₀ (the concentration of an inhibitor required to inhibit 50% of the ACE activity) were prepared an inhibition curve with using the sample concentration for X axis and ACE inhibitory activity for Y axis.

The ACE inhibition curves of L. plantarum Tensia DSM 21380 and control milk with cleaved 3-hydroxybutyric acid as product were statistically different (p<0.05). Among the two types of milk screened for ACE inhibitory activity, the control milk (acidified with lactic acid) showed maximum inhibition percentage of 82.8%, while milk fermented with L. plantarum Tensia DSM 21380 showed maximum ACE inhibition of 83.6%. Overall ACE inhibitory activity was significantly higher (p<0.05) for milk fermented with TENSIA as indicated by the lower IC₅₀ value 2.7±0.9 μM while the IC₅₀ value for the control milk was 6.6±0.3 μM.

Significantly (p<0.05) higher ACE inhibition was found in milk fermented with L. plantarum Tensia DSM 21380 in vitro in comparison with control (milk acidified with lactic acid): TENSIA IC₅₀=2.7±0.9 μM and control IC₅₀=6.6±0.3 μM (p<0.05). This indicates a possible mechanism of the L. plantarum Tensia DSM 21380 for lowering systolic and diastolic blood pressure.

Targeted metabolic profiling of cheese comprising L. plantarum Tensia DSM 21380 Cheese comprising L. plantarum Tensia DSM 21380 were analyzed in untargeted metabolic profiling as well as in targeted analysis of potential angiotensin converting enzyme (ACE) inhibitory peptides.

5 g of grated cheese was mixed with 10 ml MilliQ water and followed by shaking in an orbital shaker incubator (Biosan ES-20, Ikamag) for 60 minutes at 40° C. The samples were centrifuged at 10 000 and +4° for 30 minutes. The aqueous solution below the fat layer was removed with a syringe and 500 μl of the solution was filtered through an Amicon® Ultra 3 KDa centrifugal filter device (Millipore, Ireland Ltd) by centrifugation at 14000 g for 30 min. The composition of cheese homogenates obtained by different preparation techniques was described by untargeted qualitative mass-spectrometry analysis (Q-TRAP 3200, Applied Biosystems, USA).

### Results.

The signal of acetylcholine was found to be 4.6 fold stronger (p<0.0001) in probiotic cheese samples in comparison with control.

Additionally, the known ACE inhibitory peptides of lactobacilli origin IPP and VPP were detected in all samples of probiotic cheese comprising L. plantarum Tensia DSM 21380.

### DESCRIPTION OF THE DRAWINGS

FIG. 1. Lactobacillus plantarum Tensia DSM 21380 molecular identification by ITS-PCR

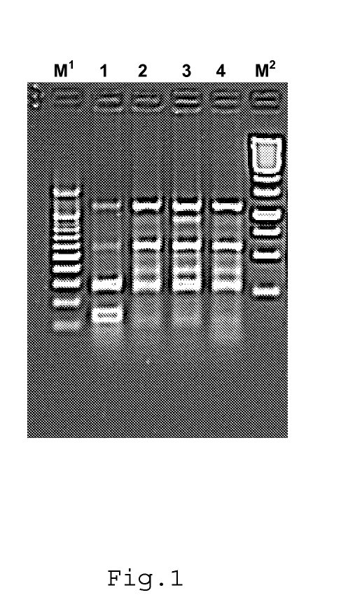

1. L. plantarum DSM 21379

2. L. plantarum Tensia DSM 21380

3. L. plantarum CRL 972 (ATCC 14917)

M-100 bp marker (Fermentas)

FIG. 2. L. plantarum Tensia DSM 21380 molecular fingerprints in comparison with L. plantarum control strain (Pulse field-gel-electrophoresis profile, PFGE).

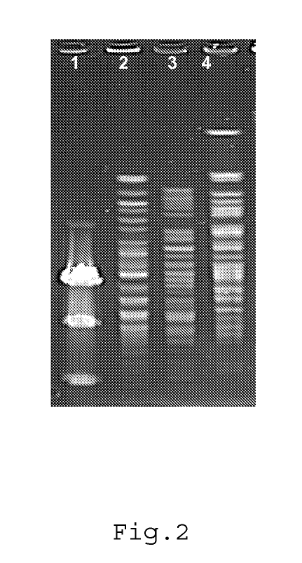

1. Lambda Ladder PFG Marker (New England Bio Labs Inc.)

2. L. plantarum Tensia DSM 21380

3. L. plantarum DSM 21379 (control)

FIG. 3. L. plantarum Tensia DSM 21380 presence in cheese by DGGE method DGGE gel-electrophoresis form test-cheeses (cheeses with probiotic additive, control cheese, cheese with prebiotic additive).

| | |
|---|---|
| 1. | Cheese nr 529 |
| 2. | Control cheese |
| 3. | Cheese nr 5 |
| 4. | L. plantarum strain no 1 |
| 5. | Cheese nr 9-1 |
| 6. | L. plantarum DSM 21379 |
| 7. | Cheese nr 19 |
| 8. | L. plantarum strain no 1 |
| 9. | Cheese nr 9-2 |
| 10. | L. plantarum DSM 21379 |
| 11. | L. plantarum Tensia DSM 21380 cheese |
| 12. | L. plantarum Tensia DSM 21380 pure culture |
| 13. | Cheese nr 4 |
| 14. | Cheese nr 12 |
| 15. | L. paracasei strain no 1 |
| 16. | L. plantarum DSM 21379 |

FIG. 4. Lactobacillus species by Pearson UPMAG cluster analyses in L. plantarum Tensia DSM 21380 group.

FIG. 5. Reduction of systolic blood pressure (end of the trial in comparison with the values of systolic blood pressure at the recruitment)

(a) Positive correlation with the minimal increase of lactobacilli counts (FISH) after the consumption of L. plantarum Tensia DSM 21380 comprising cheese (r=0.615, p=0.044, n=11)

(b) correlation with the increase of putrescine in urine of volunteers after the consumption of L. plantarum Tensia DSM 21380 comprising cheese in comparison with the values at the recruitment (r=0.631, p=0.037, n=11).

### DESCRIPTION OF THE EMBODIMENTS

### Example 1

### Suppression of NSLAB Microbes in Food Product

Test with Estonian Cheese

Method. Microorganism Lactobacillus plantarum Tensia DSM 21380 was added to the cheese milk of Dairy Cooperative E-Piim, (inoculation dose 3×10⁸ CFU/vat) and the milk was renneted (25 min). The curds were cut (25 min), heated (34° C. 15 min), dried (25 min), pressed, drained (1 h), salted in brine (12° C.; 20% NaCl; pH 4.7) 20 h, drained and dried (8 h), backed into plastic and ripened at 12° C. for at least 4 weeks.

The following illustrates survival of Lactobacillus plantarum Tensia DSM 21380 in cheese and the raise of metabolic activity of the Lactobacillus plantarum Tensia DSM 21380 during cheese ripening and shelf-life when inoculated into cheese milk in freeze-dried form (Batch nr 13).

| | |
|---|---|
| Density of L. plantarum Tensia in freeze-dried starter | 9 × 10¹⁰ CFU/g |
| Density of L. plantarum Tensia in cheese milk | 2 × 10⁴ CFU/ml |
| Density of L. plantarum | 10⁵ CFU/g |
| Tensia in fresh cheese after brine | |
| Density of L. plantarum Tensia in 15 days old cheese | 10⁶ CFU/g |
| Density of L. plantarum Tensia in 1 month old cheese | 1 × 10⁷ CFU/g |
| Density of L. plantarum | 1 × 10⁸ CFU/g |
| Tensia in 1.5 month old cheese | |
| Density of L. plantarum Tensia in 4 month old cheese | 3 × 10⁶ CFU/g |

TABLE 8
Lactobacilli content in L. plantarum Tensia DSM
21380 comprising cheese at 28^{th} day of ripening.

| | L. plantarum | | L. plantarum Tensia | | | | | |
|---|---|---|---|---|---|---|---|---|
| | Tensia | | DSM 21380 comprising | | | | | |
| | DSM21380 | Control | cheese | | | Control cheese | | |
| | comprising | cheese | | | 3 | | | 3 |
| | cheese | pH 5.1 | Day 3. | Day 28. | months | Day 3. | Day 28. | months |
| Lactobacillus sp | 10⁹ | 9 × 10⁵ | 6 × 10⁵ | 5 × 10⁸ | 5 × 10⁷ | ND | 10⁸ | 10⁷ |
| L. plantarum | 10⁹ | 2 × 10³ | 6 × 10⁵ | 5 × 10⁸ | 5 × 10⁷ | ND | ND | ND |
| L. casei | ND | 8 × 10⁵ | ND | ND | ND | ND | ND | ND |
| L. buchneri | ND | 10⁵ | ND | ND | ND | ND | ND | ND |
| OHOL* | ND | ND | ND | ND | ND | ND | 2 × 10³ | ND |

OHOL—obligatively homofermentative lactobacilli
ND—not detected

The L. plantarum counts were 10 thousand times higher in L. plantarum Tensia DSM 21380 comprising cheese to the day 28 to in comparison with the control cheese. While in control cheese the homofermentative lactobacilli, L. casei and L. buchneri strains were present, then all aforementioned species were missing in L. plantarum Tensia DSM 21380 comprising cheese. Thus the strain Lactobacillus plantarum Tensia DSM 21380 possesses the ability to inhibit cheese NSLAB and suppress the cheese contaminating microbiota. The latter can be pathogens occurring in food product after preparation. Therefore L. plantarum Tensia DSM 21380 addition could help to lengthen the shelf-life of a food product.

The antimicrobial activity of L. plantarum Tensia DSM21380 against Listeria monocytogenes in the cheese environment

The study aimed to assess the antimicrobial activity of L. plantarum Tensia DSM21380 against Listeria monocytogenes in cheese environment.

### Method 1

Six Edam-type laboratory scale experimental cheeses were prepared. Two control cheeses did not comprise microbial additives. Two cheeses comprised pathogen Listeria monocytogenes ATCC 13932 and two cheeses comprised both L. plantarum Tensia DSM21380 and Listeria monocytogenes. The pasteurised milk was inoculated with 1% of cheese starter C92 (CSK Food Enrichment, Holland) and with test-microbes, was renneted at 32° C. (cheese rennet FROMASE 2200 TL granulate). The curds were cut, heated (37-38° C.), and dried, pressed, drained and salted, coated with plastic and ripened at 15° C. for 30 days. The count of TENSIA in fresh cheese was 10⁵ CFU/g and L. monocytogenes 10⁴ CFU/g.

For microbiological analyses of cheese, samples were aseptically taken from the centre of the cheese block. 0.9% of NaCl (1:9) was added to the cheese sample, homogenized in blender (MiniMix, Interscience, France), serially diluted and 0.1 ml of each dilution was seeded onto MRS agar to detect the counts of lactobacilli. Listeria chromogenic agar medium with pre-enrichment method was used in order to assess the counts of Listeria monocytogenes (Oxoid, England).

### Assessment of the Short Chain Fatty Acid Content in the Experimental Cheeses.

10 ml of distilled water was added to cheese samples (10 g), homogenised in a blender, extracted or esterified. Acetic, propionic, butyric, valeric, capronic, succinic and lactic acid content was measured quantitatively by gas chromatograph (HP 6890 Series GC System). A capillary column HP-INNOWax (15 m×0.25 mm, 0.15 μm) was used. The column temperature program: I 60° C. 1 min, 10 min, and II 20° C./min 120° C. 80° C. 1 min, 8 min 25° C./min 120° C., detector (FID) to 250° C.

A digital pH meter E6115 (Evikon MCI, Estonia) was used for measuring the pH of the cheeses. pH was measured in three parts of a cheese block.

The counts of L. plantarum Tensia DSM21380 increased during ripening for 2 logs. No significant changes in the counts of L. monocytogenes were detected in the control cheese. In test-cheese comprising Listeria monocytogenes and L. plantarum Tensia DSM21380, the counts of L. monocytogenes decreased to very low values i.e. to 2.4 log₁₀ per 1 g of cheese (Table 8A).

TABLE 8A
Survival of Listeria monocytogenes (CFU log10/g)
in experimental cheeses during ripening

| Additive in | Count of L. monocytogenes CFU log10/g | | | |
|---|---|---|---|---|
| experimental cheese | Day 0 | Day 10 | Day 20 | Day 30 |
| L. plantarum | 3.2 ± 0.3 | 2.9 ± 0.2 | 2.6 ± 0.9 | 2.4 ± 0.6 |
| Tensia DSM21380 + | | | | |
| L. monocytogenes | | | | |
| L. monocytogenes | 4.2 ± 0.6 | 4.0 ± 0.9 | 4.8 ± 2.2 | 4.2 ± 0.2 |

Adding L. plantarum Tensia DSM21380 fostered the increase of succinic acid content during the ripening of cheeses (Table 8B).

A positive correlation between the count of lactobacilli and acetic and succinic acids and negative correlation between listeria and lactic acid appeared in the cheese comprising L. plantarum Tensia DSM21380+L. monocytogenes.

TABLE 8B
Content of short chain fatty acids (g/kg) and pH and counts of L. plantarum Tensia
DSM21380 and L. monocytogenes ATCC 13932 in 30 days ripned experimental cheeses

| | | Cheese | Cheese | |
|---|---|---|---|---|
| | | comprising | comprising | |
| | | L. plantarum | L. plantarum | Cheese |
| | | Tensia | Tensia DSM21380 + | comprising |
| Assessed parameters | | DSM21380 | L. monocytogenes | L. monocytogenes |
| Short chain | Acetic acid | 0.38 ± 0.01 | 0.3 ± 0.06 | 0.22 ± 0.18 |
| fatty acids | Propionic acid | 0.11 ± 0.00 | 0.11 ± 0.00 | 0.03 ± 0.06 |
| g/kg | Butyric acid | 0.09 ± 0.09 | 0.03 ± 0.01 | 0.02 ± 0.00 |
| | Capronic acid | 0.05 ± 0.04 | 0.03 ± 0.01 | 0.02 ± 0.01 |
| | Lactic acid | 15.37 ± 0.58 | 14.34 ± 0.88 | 14.14 ± 2.38 |
| | Succinic acid | 0.93 ± 0.20 | 0.88 ± 0.37 | 0.36 ± 0.11 |
| Survival of | L. plantarum | 7.7 ± 0.1 | 7.8 ± 0.00 | — |
| added | Tensia | | | |
| microbes | DSM21380 | | | |
| log10/g and | L. monocytogenes | — | 2.4 ± 0.6 | 4.2 ± 0.2 |
| pH | pH | 4.9 ± 0.3 | 4.8 ± 0.4 | 4.8 ± 0.2 |

By testing the antimicrobial activity of Lactobacillus sp on agar medium the tested lactobacillus strain did not got into direct contact with the pathogen or lactobacillus strain used as a target microbe, against what its antagonistic activity was tested. The antagonistic activity, registered as the growth inhibition of the pathogen was based on the metabolites (including various organic acids including acetic, lactic and succinic acid) that diffuse into the agar medium during the growth of L. plantarum Tensia DSM21380. In the foodstuff, including dairy products (cheese), the viability of non-starter lactobacilli and/or other contaminants of raw-milk origin are suppressed by the lactobacillary metabolites as well as by direct contact with the probiotic lactobacillus strain. Direct contact with the target microbe may stimulate at lower pH values caused by organic acids the synthesis of other antimicrobial compounds (bakteriocins), (Aasen I. M., Moretro T., Katla T., Axelsson L. and Storro I. (2000) Influence of complex nutrients, temperature and pH on bacteriocin production by Lactobacillus sakei CCUG 42687. Appl. Microbiol. Biotechnol. 53, 159-166.)

Short-chain fatty acid profile of L. plantarum Tensia DSM21380 and the presence of antimicrobial activity of the strain help to suppress Listeria sp. in food environment. Sustaining the antimicrobial properties in cheese matrix ensures the suppression of putative food-borne pathogens by Tensia.

Detection of Lactobacillus plantarum Tensia DSM 21380 Counts in Cheese by DGGE

Method. Microbial DNA was isolated from cheese by QIAamp DNA Mini Kit (QIAGEN) and amplified with primers 968-GC-f (GGGAACGCGAAGAACCTTA-GC), 1401-r (CGGTGTGTACAAGACCC).

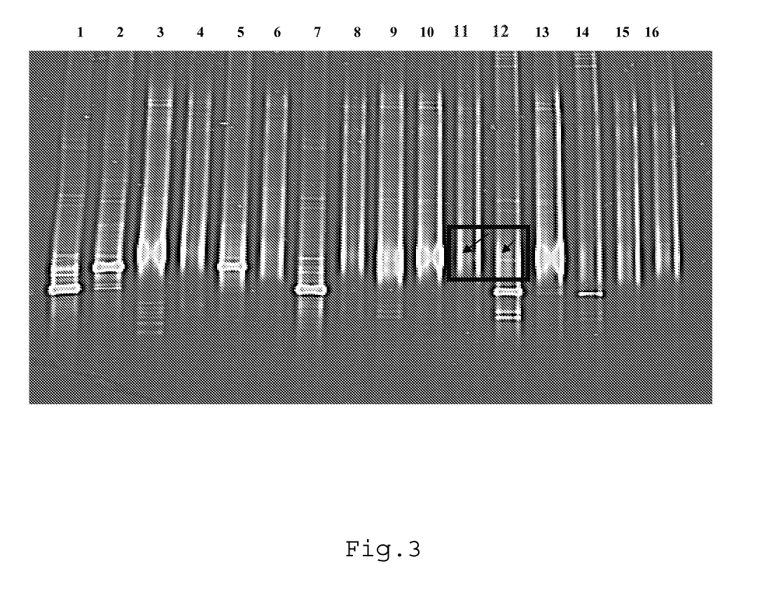

PCR products were separated by DGGE electrophoresis on a 30-60% acrylamide containing gel using Dcode™ System technique (Bio-Rad, Hercules, Calif.) (FIG. 3).

Detection of Biogenic Amines in L. plantarum Tensia DSM 21380 Comprising Cheese

Method. Cheese samples were extracted (20 ml 50% methanol solution was added to 10 g of cheese and incubated at 45° C. for 1 h, cooled to 30° C. and centrifuged) and 200 μl of upper layer was derivatized for GC analyze by modified method of Nakovichi (Nakovich, L. 2003 Analysis of biogenic amines by GC/FID and GC/MS) in Department of Microbiology of the University of Tartu.

GC analysis were carried out by gas chromatograph HP 6890 Series GC System, with capillary colonna HP-5 19091J-413 (30 m×0.32 mm; 0.25 μm). The column temperature program 160° C. 1 min, 20° C./min 280° C. 15 min; and detector (FID) 350° C.

TABLE 9
Biogenic amines and polyamines in L. plantarum Tensia DSM
21380 comprising test-cheeses from industrial test trials

| | Viable counts of strain | | | | Viable count of |
|---|---|---|---|---|---|
| | incorporated into | | | | L. plantarum |
| | cheese (CFU/g) at day | Amines (mg/kg) | | | Tensia DSM 21380 |
| Sample | 3-4 after preparation | Tyramine | Putrescine | Cadaverine | in ripe cheese |
| L. plantarum | 6.5 × 10⁸ | 0.69 | 1.32 | 0 | 10⁶ |
| Tensia DSM | | | | | |
| 21380, 1. Batch | | | | | |
| Control cheese | — | 2.31 | 1.82 | 0 | — |
| 1. Batch | | | | | |
| L. plantarum | 4.5 × 10⁶ | 2.65 | 7.46 | 0 | 6 × 10⁸ |
| Tensia DSM | | | | | |
| 21380, 2. Batch | | | | | |
| Control cheese | | 5.64 | 1.84 | 0 | 10⁷ |
| 2. Batch | | | | | |
| L. plantarum | 2 × 10⁶ | 5.49 | 7.29 | 0 | 10⁸ |
| Tensia DSM | | | | | |
| 21380, 3. Batch | | | | | |
| Control cheese | — | 0 | 0 | 0 | — |
| 3. Batch | | | | | |

L. plantarum Tensia DSM 21380 produced tyramine 0.69-5.49 mg per kg of cheese and putrescine in lower quantities: 1.32-7.46 mg/kg.

### Example 2

### Lactobacillus plantarum Tensia DSM 21380 Safety Trial with NIH Mice

For the acceptance by the Estonian Veterinary and Food Board, the safety of probiotic strains and the food containing theses strains should be tested.

Method. In the experimental model with NIH mice 3 groups of mice consumed different cheeses during 30 days (control cheese with no additives, Lactobacillus plantarum Tensia DSM 21380 comprising cheese). Mice stayed in good condition, cheese administration caused increase of body weight, no changes in fur and digestion was detected. No translocation of administrated strains or other microbes into blood or organs was detected. No pathological shifts were found by the morphological and histological evaluation of the liver and spleen which proves the safety of Lactobacillus plantarum strain Tensia DSM 21380.

### Example 3

### Effect of Lactobacillus plantarum Tensia DSM 21380 Comprising Cheese on Blood Indices and Intestinal Microflora of Healthy Volunteers

The aim of the clinical trial (randomized blinded cross-over placebo controlled) was to evaluate the safety and effect on intestinal microflora of cheese comprising antimicrobial Lactobacillus plantarum Tensia DSM 21380 on healthy volunteers.

Persons and Methods: Participants were healthy volunteers, both male and female 12 persons (M/F 5/7; 21-43 years). For exclusion criteria diabetes, glucose and glycohemoglobin HbAlc from blood sera were detected.

Test cheeses comprised strain Lactobacillus plantarum Tensia DSM 21380 (viable counts of 5×10⁸ CFU/g cheese). Before consumption the test-cheese was incubated with Lactobacillus plantarum Tensia DSM 21380 for 30 days at 12° C. Regular Estonian cheese without additives served as a control. Trial was a randomized blinded cross-over placebo controlled trial. Trial started with 3-week consumption of test cheese, followed by 2 week washout period, after which the control cheese was consumed for 3 weeks. Dose 50 g/day.

TABLE 10
Clinical data of healthy volunteers after consumption of probiotic
cheese comprising L. plantarum Tensia DSM 21380

| | Probiotic cheese with | | | | |
|---|---|---|---|---|---|
| | L. plantarum Tensia DSM 21380 | | Control cheese | | |
| | | After | | After | |
| | Baseline | treatment | Baseline | treatment | P values |
| BMI | 24.1 ± 3.6 | 24.2 ± 3.6 | 23.8 ± 3.5 | 23.9 ± 3.6 | 0.625/0.399 |
| (kg/m²) | | | | | |
| Systolic | 112.9 ± 10.4 | 107.1 ± 10.4 | 110.3 ± 8.3 | 109.3 ± 9.4 | 0.016/0.655 |
| pressure | | | | | |
| (mm Hg) | | | | | |
| Diastolic | 78.8 ± 7.1 | 74.3 ± 8.8 | 74.8 ± 5.2 | 75.7 ± 8.3 | 0.021/0.668 |
| pressure | | | | | |
| (mm Hg) | | | | | |

Body mass index (BMI) (kg/m²): 19-25 kg/m² - normal, 26-30 kg/m² - overweight, over 30 - obesity.

Both systolic and diastolic pressure were significantly lower after 3-week consumption of probiotic L. plantarum Tensia DSM 21380 (5×10⁸CFU/g×50 g) comprising cheese. The 3-week consumption of strain Tensia DSM 21380 comprising cheese did not increase the body mass index.

Effect of Lactobacillus plantarum Tensia DSM 21380 on Intestinal Microflora

Method. Quantitative analysis of fecal microflora and species of lactobacilli, methods elaborated in Department of Microbiology of Tartu University were (Mikelsaar M E, Valjaots M E, Lenzner A A. Anaerobe Inhalts-und Wandmikroflora des Magen-Darm-Kanals. Die Nahrung, 1984, 23, 6/7, 727-733; Sepp E., Julge K., Vasar M., Naaber P., Björksten B., Mikelsaar M. Intestinal microflora of Estonian and Swedish infants. Acta Paediatrica, 1997, 86, 956-961).

TABLE 11
Lactobacilli counts in the faeces of healthy volunteers (log₁₀ cfu/g faeces)

| | L. plantarum Tensia DSM | | | | P values |
|---|---|---|---|---|---|
| | 21380 comprising cheese | | Control cheese | | paired t-test |
| | BL1 | PRO | BL2 | PL | BL1 vs |
| | mean ± stdev | mean ± stdev | mean ± stdev | mean ± stdev | PRO/BL2 |
| | range (median) | range (median) | range (median) | range (median) | [symbol] |
| LB total count | 5.1 ± 1.9 | 6.7 ± 1.0 | 5.7 ± 1.4 | ND | 0.025/ |
| cultivation | 0-6.3 (5.9) | 4.9-8.6 (6.7) | 4.0-8.6 (5.3) | | |
| LB total count | 8.4 ± 0.1 | 8.4 ± 0.3 | 8.4 ± 0.3 | 8.3 ± 0.3 | 0.748/0.244 |
| FISH | 8.1-8.6 (8.4) | 7.9-9.1 (8.3) | 8.0-8.8 (8.3) | 7.6-9.0 (8.2) | |
| L. plantarum * | 0-5.3 (0) | 0-8.6 (5.9) | 0-4.3 (0) | ND | 0.006/ND |
| range/median | 3/12 | 10/12 § | 2/12 | | |
| prevalence | | | | | |

ND—not determined [symbol]
indicates data missing or illegible when filed

The total count of lactobacilli and the prevalence of L. plantarum*increased (p=0.006) in faeces of volunteers.

TABLE 12
Prevalence of L. plantarum as species in fecal samples
of volunteers at the recruitment (BL1), after probiotic
cheese consumption (PRO) and recovery (BL 2)

| Group | BL 1 | PRO | BL 2 |
|---|---|---|---|
| Group 1 (n = 12) | 3/12 | 10/12 | 2/12 |
| Group 2 (n = 12) | 2/12 | 3/12 | 4/12 |

Increase of L. plantarum as species was detected (Fischer exact test, 3/12 vs 10/12, p=0.006) in the L. plantarum Tensia DSM 21380 group.

Thus, L. plantarum Tensia DSM 21380 affects positively human GIT microflora though increase of beneficial lactobacilli counts.

Surprisingly, by increasing the counts of facultative heterofermentative lactobacilli (FHEL; 4.7±1.3 vs. 5.7±1.3, p=0.029), the fermentation group, where L. plantarum Tensia DSM 21380 belongs, the increase of obligatively homofermentative lactobacilli (OHOL) count was detected (4.6±1.1 vs. 5.7±1.5, p=0.014). Thus, L. plantarum Tensia DSM 21380 helps significantly to stabilize GT lactoflora.

Fecal Lactobacilli Counts of Volunteers by Molecular Methods (fluorescent In Situ Hybridization, FISH)

Method. Fecal samples were diluted to 1/10 in PBS buffer. Microbial cells were fixed with 4% formaldehyde solution and kept at 4° C. FISH was carried out by method of Harmsen with the probe Lab 158 tagged with dye Cy 3. Tagged microbial cells were counted fluorescence microscopicy.

Results are presented in table 11 together with the results of bacteriological analyses. According to FISH, (the method that registers also dead cells) the lactobacilli counts remained practically unchanged.

The lactobacilli DGGE profile from faeces of L. plantarum Tensia DSM 21380 comprising volunteers that consumed cheese.

Method. Subsequently, the PCR product was separated by DGGE electrophoresis in 30-60% acrylamide containing gel with Dcode™ System technique (Bio-Rad, Hercules, Calif.). Gels were analyzed by BioNumerics 2.5 (Applied Maths, Belgium) software according to Peasoni correlation (Heilig H G, Zoetendal E G, Vaughan E E, Marteau P, Akkermans, A D L, de Vos W M. Molecular diversity of Lactobacillus ssp. and other Lactic acid bacteria in the human intestine as determined by specific amplification of 16S ribosomal DNA. Appl Envir Microbiol 2002; 68: 114-123).

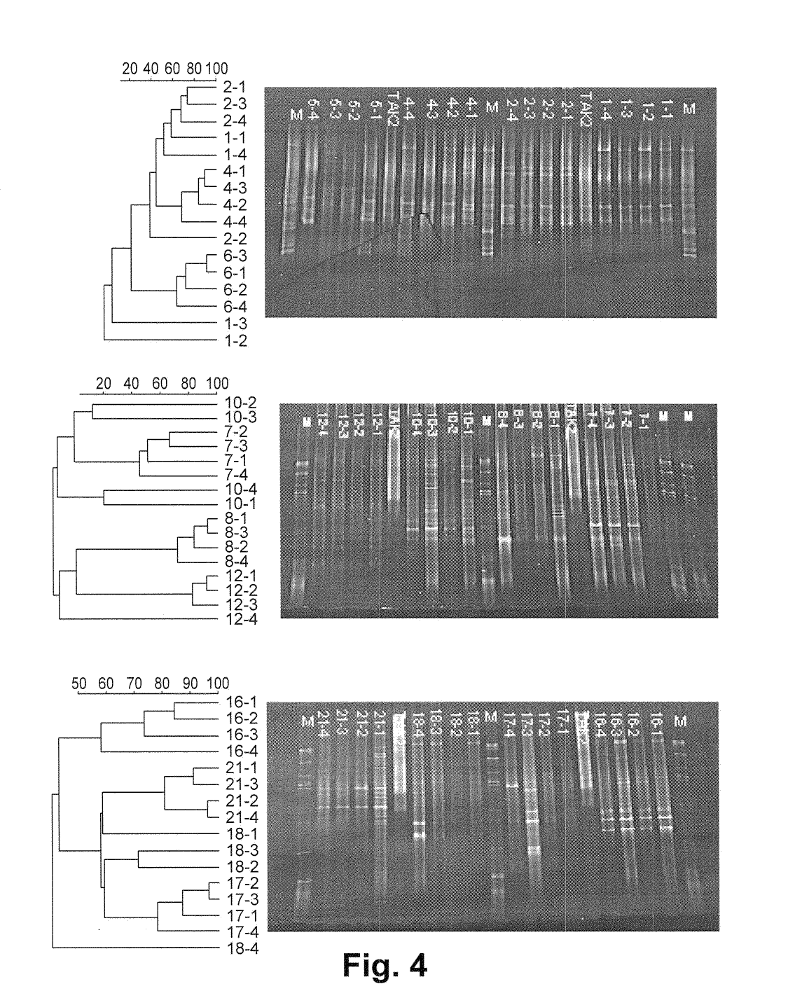

Gel photos (FIG. 4) indicate profile of dominant lactobacilli species in the subject's faeces according to 16S rDNA amplification. With the UMPAG software it was possible to analyze putative matrix coincidence between different gels, i.e. to detect significant difference (>20%) between different gels/different analysis.

TABLE 13
Changes in lactoflora in L. plantarum Tensia DSM 21380 group
during the clinical trial

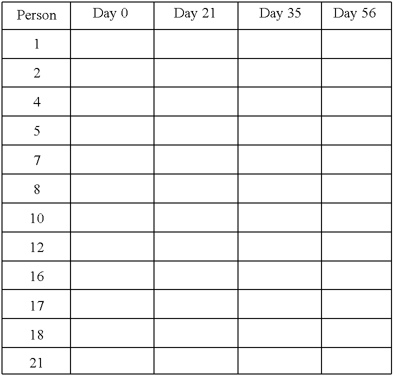

Table 13 indicates, that in the L. plantarum Tensia DSM 21380 group the indigenous lactoflora pattern changed in 10 persons from 12 to the day 21 (p<0.05). According results were obtained in L. plantarum colonization survey by bacteriological method. The changes remained stable in 4 persons even after 2 weeks.

### the Polyamines and Biogenic Amines Content in the Urine of Volunteers

For the evaluation of biogenic amines before and after L. plantarum Tensia DSM 21380 comprising cheese consumption and at the stabilization period, the morning urine and gas chromatography method were used.

Method. Urine samples were derivatized with propylchlorophormate for GC analyze by modified method of Ugland (Ugland H G; Krough M, Rasmussen K E: Aqueous alkylchloroformate derivatization and solid-phase microextraction: determination of amphetamines in urine by capillary gas chromatography. J Chromatography B Biomed Sci Appl 1997; 701:29-38).

GC analysis were carried out by gas chromatograph HP 6890 Series GC System (Hewlett Packard, Avondale, Pa., USA), with capillar colonne HP-5 19091J-433 (30 m×0.25 mm; 0.25 μm) The column temperature program 150° C. 1 min, 20° C./min 280° C. for 5 min; and detector (FID) 250° C. The biogenic amines concentration was calculated according to nmol/mol creatinine

TABLE 14
Polyamines in volunteers consuming L. plantarum Tensia DSM
21380 cheese assessed in morning urine (nmol/mol creatinine)

| | Probiotic cheese with | | | | |
|---|---|---|---|---|---|
| | L. plantarum Tensia DSM | | | | |
| | 21380 additive | | Control cheese | | P values |
| | BL1 | PRO | BL2 | PL | paired t-test |
| | mean ± stdev | mean ± stdev | mean ± stdev | mean ± stdev | BL1 vs PRO/ |
| | range (median) | range (median) | range (median) | range (median) | BL2 vs PL |
| Put | 0.110 ± 0.139 | 0.090 ± 0.120 | 0.073 ± 0.110 | 0.027 ± 0.024 | 0.496/0.275 |
| | 0-0.467 (0.046) | 0-0.396 (0.041) | 0-0.384 (0.037) | 0-0.055 (0.037) | |
| acPut | 0.607 ± 0.558 | 0.567 ± 0.431 | 0.817 ± 1.027 | 0.573 ± 0.391 | 0.510/0.677 |
| | 0-2.159 (0.478) | 0.191-1.758 (0.430) | 0.091-3.916 (0.550) | 0.182-1.403 (0.486) | |
| DAP | 0.148 ± 0.125 | 0.056 ± 0.084 | 0.090 ± 0.163 | 0.061 ± 0.099 | 0.016/0.844 |
| | 0-0.344 (0.159) | 0-0.205 (0) | 0-0.432 (0) | 0-0.246 (0) | |
| acSpd | 0.181 ± 0.137 | 0.227 ± 0.142 | 0.197 ± 0.148 | 0.258 ± 0.221 | 0.016/0.244 |
| | 0-0.428 (0.144) | 0.053-0.493 (0.182) | 0.039-0.495 [symbol] .195) | 0.041-0.686 (0.186) | |
| Cad | 0.012 ± 0.026 | 0.038 ± 0.062 | 0.041 ± 0.077 | 0.015 ± 0.037 | 0.125/0. [symbol] |
| | 0-0.084 (0) | 0-0.156 (0) | 0-0.248 (0) | 0-0.123 (0) | |

BL1—baseline 1, at the recruitment, PRO—after probiotic treatment, BL 2—baseline 2, recovery Put—putrescine, acPut—N-acetylputrescine, DAP—1.3-diaminopropane, acSpd—N8-acetylspermidine, Cad—cadaverine [symbol]
indicates data missing or illegible when filed

Consumption of L. plantarum Tensia DSM 21380 comprising probiotic cheese increased the acetylated spermidine in urine of volunteers at the end of probiotic period indicating the improvement of the metabolism of polyamines in blood and tissues. DAP concentration in urine decreased.

### Correlations

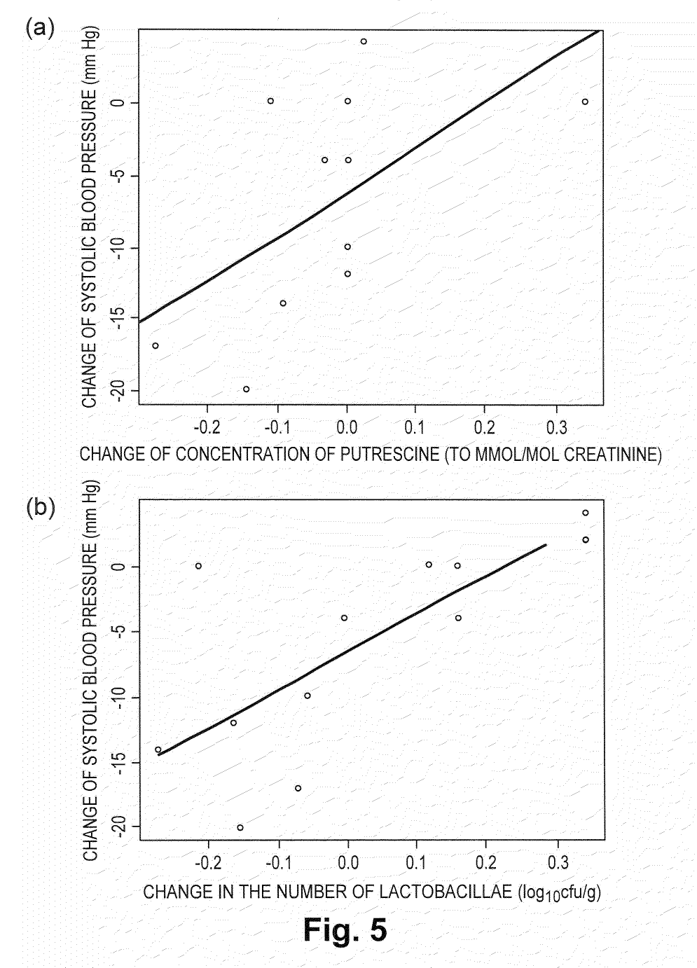

It was found that the reduction of systolic blood pressure was correlated to the increase of lactobacilli counts and the increase of putrescine in urine (FIG. 5).

### Safety

After cheese trial with volunteers, the values of systemic inflammation markers (U-CRP, ultrasensitive CRP, and leucocytes) were not changed and were within the normal range (Table 15). No change was detected also in the values of essential allergy marker IgE.

The consumption of probiotic cheese did not cause changes in WBC counts (leucogram): the proportion of cells at the end of the trial remained unchanged in comparison with the recruitment values.

TABLE 15
Inflammation markers at the beginning and the end of the trial

| | Probiotic cheese with | | | | | |
|---|---|---|---|---|---|---|
| | L. plantarum Tensia | | | | | |
| | DSM 21380 | | Control cheese | | P | Standard |
| | before | after | before | after | values | values |
| U-CRP, mg/L | 1.1 ± 0.6 | 1.0 ± 0.3 | 1.4 ± 0.9 | 1.6 ± 1.3 | 0.3/0.9 | <5 mg/L |
| Leucocytes | 5.2 ± 0.8 | 5.6 ± 1.3 | 5.1 ± 1.1 | 5.5 ± 1.1 | 0.6/0.14 | 4-10 × 10⁹/L |
| total count × 10⁹/L | | | | | | |

Consumption of L. plantarum Tensia DSM 21380 comprising probiotic cheese did not cause abdominal discomfort (abdominal pain, flatulence, bloating) or the increase of the body mass index, glucose and lipid levels.

Thus in healthy subjects the consumption of Lactobacillus plantarum Tensia DSM 21380 comprising cheese does not cause systemic inflammation, allergic sensibilisation nor does it cause harm to essential organs.

TABLE 15A
Blood serum biochemical parameters of healthy volunteers after
the consumption of cheese comprising L. plantarum DSM 21380

| | | | | | P values |
|---|---|---|---|---|---|
| | Probiotic cheese | | Control cheese | | (BL1 vs BL2, |
| | BL1 | PRO | BL2 | PL | PRO vs PL) |
| Glucose, | 4.5 ± 0.7 | 4.6 ± 0.5 | 4.6 ± 0.6 | 4.7 ± 0.5 | 0.922/0.289 |
| mmol/l | 2.8-5.5 (4.5) | 3.6-5.5 (4.5) | 3.9-6.0 (4.5) | 3.8-5.9 (4.8) | (1.0, 0.48) |
| total | 4.6 ± 0.9 | 4.6 ± 1.1 | 4.2 ± 0.6 | 4.5 ± 0.9 | 0.828/0.102 |
| cholesterol, | 3.2-6.6 (4.55) | 3.1-7.2 (4.4) | 3.1-5.1 (4.1) | 2.8-6.3 (4.4) | (0.003, 0.671) |
| mmol/1 | | | | | |
| HDL- | 1.7 ± 0.5 | 1.7 ± 0.3 | 1.6 ± 0.4 | 1.7 ± 0.4 | 0.628/0.433 |
| cholesterol, | 1.0-2.6 (1.7) | 1.1-2.3 (1.6) | 0.9-2.3 (1.7) | 1.2-2.5 (1.6) | (0.444, 0.623) |
| mmol/l | | | | | |
| LDL- | 2.7 ± 0.8 | 2.8 ± 1.1 | 2.6 ± 0.7 | 2.6 ± 0.7 | 0.296/0.827 |
| cholesterol, | 1.4-4.3 (2.7) | 1.5-5.7 (2.5) | 1.5-3.7 (2.4) | 1.2-4.3 ( 2.5) | (0.336, 0.271) |
| mmol/l | | | | | |
| Triglycerides, | 1.0 ± 0.6 | 1.0 ± 0.5 | 0.9 ± 0.4 | 1.2 ± 0.7 | 0.978/0.140 |
| mmol/l | 0.5-2.8 (0.8) | 0.4-2.1 (1.0) | 0.5-1.8 (0.8) | 0.5-2.6 (0.9) | (0.306, 0.428) |

The consumption of cheese comprising L. plantarum TENSIA DSM 21380 did not cause unwanted changes in the glucose content in blood serum or in lipid metabolism parameters (Table 15A). Values of total cholesterol and cholesterol fractions (HDL-cholesterol, LDL-cholesterol and triglycerides) remained within normal values.

### Example 4

### Effect of Lactobacillus plantarum Tensia DSM 21380 Comprising Cheese on Blood Indices of Elderly Healthy Individuals

During the clinical trial (randomized blinded cross-over placebo controlled) the safety and effect on blood indices of Lactobacillus plantarum Tensia DSM 21380 comprising cheese on elderly healthy volunteers was evaluated.

Persons and Methods. Participants were healthy volunteers, both male and female 21 persons (M/F 2/19; 61-84 years). For exclusion criteria diabetes, glucose and glycohemoglobin HbAlc from blood sera were detected.

Test cheeses comprised strain Lactobacillus plantarum Tensia DSM 21380 (viable counts of 2×10⁷ CFU/g cheese). Before consumption the test-cheese was incubated with Lactobacillus plantarum Tensia DSM 21380 for 30 days at 12° C. Regular Estonian cheese without additives served as a control. Trial was a randomized blinded cross-over placebo controlled trial. Trial started with 3-week consumption of test-cheese, followed by 2 week washout period, after which the control-cheese was consumed for 3 weeks. Dose 50 g/day.

TABLE 16
Clinical data of elderly healthy volunteers after consumption
of probiotic L. plantarum Tensia DSM 21380 comprising cheese

| | Probiotic cheese with | | | | |
|---|---|---|---|---|---|
| | L. plantarum | | | | |
| | Tensia DSM 21380 | | Control cheese | | |
| | | After | | After | |
| | Baseline | treatment | Baseline | treatment | P values |
| BMI | 27.6 ± 4.1 | 27.5 ± 4.2 | 27.5 ± 4.0 | 27.6 ± 4.2 | 0.723/0.793 |
| (kg/m²) | | | | | |
| Systolic | 138.1 ± 16.6 | 132.2 ± 16.2 | 138.7 ± 21.4 | 135.2 ± 21.1 | 0.038/0.185 |
| pressure | | | | | |
| (mm Hg) | | | | | |
| Diastolic | 77.2 ± 7.7 | 73.1 ± 8.0 | 76.4 ± 8.9 | 74.8 ± 9.2 | 0.004/0.246 |
| pressure | | | | | |
| (mm Hg) | | | | | |

Body mass index (BMI) (kg/m²): 19-25 kg/m² - normal, 26-30 kg/m² - overweight, over 30 - obesity.

The decrease of blood pressure (both systolic and diastolic) was detected on elderly subjects after the 3-week consumption of probiotic L. plantarum Tensia DSM 21380 (2×10⁷/g×50 g) comprising cheese. At the same time the consumption of cheese of relatively high fat content did not increase the body mass index of elderly volunteers (Table 16).

### Safety

The consumption of L. plantarum Tensia DSM 21380 comprising probiotic cheese by elderly did not cause abdominal discomfort (abdominal pain, flatulence, bloating).

After the cheese trial the values of systemic inflammation markers (U-CRP, and leucocytes) of the participants were not increased and were within the normal range (Table 17).

TABLE 17
The inflammation markers of blood of the elderly healthy
volunteers at the recruitment and at the end of the trial

| | Probiotic L. plantarum | | | | | |
|---|---|---|---|---|---|---|
| | Tensia DSM 21380 | | | | | |
| | comprising cheese | | Control cheese | | P | Standard |
| | before | after | before | after | values | values |
| U-CRP, mg/L | 0.13/0.81 | 0.13/0.81 | 0.13/0.8 | 0.13/0.81 | 0.13/0.81 | <5 mg/L |
| Leucocytes | 5.1 ± 1.3 | 4.9 ± 1.3 | 5.0 ± 1.0 | 5.0 ± 1.6 | 0.33/0.33 | 4-10 × 10⁹/L |
| total count × 10⁹/L | | | | | | |

No abnormalities were detected also in the values of essential allergy marker IgE or the kidney and liver markers (serum creatinine, albumine, alanine transaminase (ALAT), aspartate transaminase (ASAT)).

Comparing the total cholesterol levels of the treatment and placebo period, significant decrease was detected after the consumption of the L. plantarum Tensia DSM 21380 comprising cheese. The raise of the blood serum glucose content by 0.3 units after the probiotic cheese consumption was an undesirable effect, but in comparison with the placebo period the change was not statistically significant (Table 18) and remained within the normal range.

TABLE 18
Blood serum biochemical parameters of elderly healthy volunteers after
consumption of L. plantarum Tensia DSM 21380 comprising cheese.

| | | | | | P values |
|---|---|---|---|---|---|
| | Probiotic cheese | | Control cheese | | (BL1 vs BL2, |
| | BL1 | PRO | BL2 | PL | PRO vs PL) |
| Glucose, | 5.1 ± 0.5 | 5.4 ± 0.5 | 5.3 ± 0.5 | 5.4 ± 0.4 | 0.036/0.144 |
| mmol/l | | | | | (0.212, 0.757) |
| Total cholesterol, | 5.7 ± 0.8 | 5.6 ± 0.8 | 5.9 ± 0.9 | 5.7 ± 0.8 | 0.343/0.198 |
| mmol/l | | | | | (0.044, 0.087) |
| HDL- cholesterol, | 1.7 ± 0.4 | 1.6 ± 0.4 | 1.7 ± 0.4 | 1.7 ± 0.5 | 0.411/0.514 |
| mmol/1 | | | | | (0.073, 0.118) |
| LDL- cholesterol, | 3.9 ± 0.8 | 3.8 ± 0.7 | 4.1 ± 0.9 | 3.8 ± 0.7 | 0.557/0.052 |
| mmol/l | | | | | (0.075, 0.9187) |
| Triglycerides, | 1.1 ± 0.6 | 1.1 ± 0.6 | 1.2 ± 0.5 | 1.1 ± 0.5 | 0.380/0.394 |
| mmol/l | | | | | (0.411, 0.097) |

Thus in elderly healthy individuals the consumption of Lactobacillus plantarum Tensia DSM 21380 comprising cheese does not cause systemic inflammation, allergic sensibilisation nor cause harm to essential organs.

TABLE 18A
Biogenic amines in elderly volunteers in morning urine (nmol/mol creatinine) during
the study after the consumption of cheese comprising L. plantarum Tensia DSM21380

| | Probiotic treatment | | Control period | | P valuse |
|---|---|---|---|---|---|
| | BL1 | PRO | BL2 | PL | paired t-test |
| | mean ± stdev | mean ± stdev | mean ± stdev | mean ± stdev | BL1 vs PRO/BL2 vs PL |
| | range (median) | range (median) | range (median) | range (median) | (BL1 vs BL2, PRO vs PL) |
| Cadaverine | 0 | 0 | 0 | 0 | NA |
| Tyramine | 0.006 ± 0.028 | 0.010 ± 0.044 | 0.061 ± 0.274 | 0.001 ± 0.005 | 1.0/0.37 |
| | 0-0.129 (0) | 0-0.201 (0) | 0-1.256 (0) | 0-0.025 (0) | (1.0, 1.0) |
| Histamine | 0 | 0 | 0 | 0 | NA |

BL1—baseline 1, at the recruitment, PRO—after probiotic treatment, BL 2—baseline 2, recovery, PL—after placebo treatment

Consumption of cheese comprising L. plantarum TENSIA did not cause the raise of biogenic amines (cadaverine, histamine, tyramine) concentration in urine of the elderly volunteers (Table 18A)

The consumption of cheese comprising L. plantarum TENSIA in daily dose of 1.5×10⁸ CFU increased the count of cultivable lactobacilli (5.2 log₁₀ CFU/g at the recruitment vs 6.9 log₁₀ CFU/g after 3-week probiotic treatment, p=0.012) (Table 18B).

TABLE 18B
Lactobacilli counts (mean ± stdev/range/median) in faeces of elderly volunteers
during the study after the consumption of cheese comprising L. plantarum Tensia DSM21380

| | Probiotic treatment | | Control period | | P values |
|---|---|---|---|---|---|
| | BL1 | PRO | BL2 | PL | paired t-test |
| | mean ± stdev | mean ± stdev | mean ± stdev | mean ± stdev | BL1 vs PRO/BL2 vs PL |
| | range (median) | range (median) | range (median) | range (median) | (BL1 vs BL2, PRO vs PL) |
| Total | 5.2 ± 2.7 | 6.9 ± 1.4 | 5.4 ± 2.9 | 6.1 ± 2.0 | 0.012/0.252 |
| lactobacilli | 0-8.3 (6.3) | 4.3-10.0 (6.3) | 0-9.7 (5.7) | 0-9.1 (6.5) | (0.850, 0.245) |
| CFU/g | | | | | |
| L. plantarum | 3.1 ± 3.2 | 5.2 ± 2.9 | 1.1 ± 3.0 | 3.2 ± 3.3 | 0.079/0.119 |
| | 0-8.3 (3.8) | 0-8.3 (6.2) | 0-9.6 (0.0) | 0-9.1 (3.5) | (0.083, 0.222) |
| L. plantarum | — | 5.3 ± 2.9 | ND | | |
| TENSIA* | | 0-8.3 (6.3) | | | |

*Identification with real-time PCR according to L. plantarum Tensia strain specific sequence

The probiotic treatment increased the prevalence of the L. plantarum in faeces (10/21 vs 17/21 persons), but the change was not significant. However, the increase in L. plantarum counts was in negative correlation with the decrease of systolic blood pressure (r=−0.463, p=0.046) during probiotic cheese treatment.

L. plantarum TENSIA was detectable in 81% of study participants with molecular methods in faecal samples after the probiotic treatment. However, after 2-week washout period the strain was not detectable in faeces of participants by conventional cultivation method combined with RAPD-PCR.

After the 3-week consumption of cheese comprising L. plantarum TENSIA no significant change in the polyamines content in morning urine of volunteers was detected, though there was an increasing tendency in the values of putrescine (Table 18C). However, the decrease of systolic blood pressure was correlated with the increase of putrescine (r=−0.448, p=0.042)

TABLE 18C
Polyamines in elderly volunteers in morning urine (nmol/mol creatinine) during the
study after the consumption of cheese comprising L. plantarum Tensia DSM21380

| | Probiotic treatment | | Control period | | |
|---|---|---|---|---|---|
| | BL1 | PRO | BL2 | PL | P values |
| | mean ± stdev | mean ± stdev | mean ± stdev | mean ± stdev | BL1 vs PRO/BL2 vs PL |
| | range (median) | range (median) | range (median) | range (median) | (BL1 vs BL2, PRO vs PL) |
| Put | 0.87 ± 0.78 | 0.94 ± 0.76 | 1.63 ± 2.57 | 1.16 ± 1.62 | 0.452/0.137 |
| | 0-3.30 (0.645) | 0.071-2.36 (0.565) | 0.233-12.43 (0.928) | 0.14-7.81 (0.717) | (0.065, 0.759) |
| acPut | 2.58 ± 5.72 | 1.96 ± 2.53 | 1.48 ± 1.89 | 3.50 ± 6.48 | 0.609/0.374 |
| | 0.17-26.0 (0.82) | 0.23-10.2 (1.101) | 0.15-7.71 (0.64) | 0.13-23.70 (0.72) | (0.973, 0.633) |
| acSpd | 0.02 ± 0.10 | 0.02 ± 0.10 | 0.06 ± 0.28 | 0.06 ± 0.27 | 1.0/1.0 |
| | 0-0.47 (0) | 0-0.47 (0) | 0-1.30 (0) | 0-1.24 (0) | (1.0, 1.0) |

BL1—baseline 1, at the recruitment, PRO—after probiotic treatment, BL 2—baseline 2, recovery, PL—after placebo treatment, Put—putrescine, acPut—N-acetylputrescine, acSpd—N8-acetylsperm
Conclusion: The blood pressure lowering effect of cheese comprising L. plantarum Tensia DSM21380 may have potential implications for the blood pressure management of elderly people.

### Example 5

### Sustained Blood Pressure Lowering Effect of the Consumption of Lactobacillus plantarum Tensia DSM 21380 Comprising Cheese

The DBPC parallel-designed two-armed study ISRCTN29105501 aimed to investigate the short termed (4 weeks) and the long-termed (8 weeks) effect of the consumption of the Edam-type semi-hard cheese comprising L. plantarum Tensia DSM 21380.

The daily dose of L. plantarum Tensia DSM 21380 administered with 50 g cheese was 5×10⁹ CFU. The study group consisted of 118 adults with high-normal blood pressure. Continuous consumption of the semi-hard cheese with L. plantarum Tensia DSM 21380 over longer period (8 weeks) resulted in significantly different reduction (p=0.007) in systolic blood pressure (SBP) accompanied by the significantly different reduction (p=0.026) in diastolic blood pressure (DBP) in verum group as compared to the control.

The significant reduction of both SBP and DBP throughout the study occurred within the verum group (p=0.006 in week 4 vs start and p=0.005 in week 4 vs week 8 for SBP, respective p values for DBP being 0.033 and <0.001). The average reduction of SBP during 8 weeks of administration of the cheese comprising L. plantarum Tensia DSM 21380 was 6.66 mmHg and 4.34 mmHg for DBP. The corresponding figures for placebo were respectively 2.56 mmHg and 1.21 mmHg.

### Example 6

### The Effect of the Yoghurt Containing Lactobacillus plantarum Tensia DSM 21380 on the Parameters of Blood Serum and Intestinal Microflora of Healthy Volunteers

The objective of the clinical trial (cross-over randomised placebo controlled double-blind trial) was to assess the safety and effect of the yoghurt containing Lactobacillus plantarum Tensia DSM 21380 on the parameters of blood serum and intestinal microflora of healthy volunteers (international registration number of the clinical trial ISRCTN68198472).

Subjects and methods. The participants were 25 healthy volunteers of both sexes (M/F 9/16; 31.4±10.0 a). Glucose and glycohemoglobin (HbAlc) was deteced form the blood serum in order to exclude possible latent diabetes.

Test-yoghurt contained the strain Lactobacillus plantarum Tensia DSM 21380 (5×10⁶-10⁷ microbial cells/ml). Yoghurt without probiotic additive served as control. The trial started with 3-week consumption of test-yoghurt. The washoutperiod followed, arter what the study participants consumed control yoghurt for 3 weeks. Daily dose was 10⁸-5×10⁹ microbial cells.

Survival of Lactobacillus plantarum Tensia DSM 21380 in yoghurt and raise of metabolic activity of the Lactobacillus plantarum Tensia DSM 21380 in yoghurt and shelf-life when inoculated into yoghurt milk in freeze-dried form.

Test yoghurt was prepared using the freeze-dried culture of Lactobacillus plantarum Tensia as probiotic adjunct starter in the dose of 1 g/1 ton into milk. The yoghurt was prepared traditionally. The yoghurt was packed into cups of 150 g and stored at +4° C. The survival of the probiotic strain Lactobacillus plantarum Tensia was assessed during the shelf life of the yoghurt.

| | L. plantarum Tensia (CFU/g) |
|---|---|
| freeze-dried starter | 1 × 10¹¹ |
| Day 7 after preparation | 1 × 10⁶ |
| Day 22 after preparation | 2 × 10⁶ |
| Day 26 after preparation | 2 × 10⁶ |

### Safety

After completion of the trial the important human systemic inflammation markers (ultrasensitive C-reactive protein and total count of leucocytes) were not changed and were within normal range (Table 19). No anomalies were observed in the essential allergy marker Ige

TABLE 19
Inflammation markers before and at the end of the trial

| | L. plantarum Tensia | | | | P values | |
|---|---|---|---|---|---|---|
| | DSM 21380 | | | | baseline vs. | |
| | containing probiotic | | | | probiotic | |
| | yoghurt | | Control yoghurt | | baseline vs. | Standard |
| | Before | After | Befoer | After | control yoghurt | values |
| U-CRP, mg/L | 0.7 ± 1.0 | 1.1 ± 1.3 | 1.,2 ± 1.5 | 1.2 ± 1.4 | 0.782/0.306 | <5 mg/L |
| Leucocytes | 5.8 ± 1.2 | 6.2 ± 1.3 | 6.0 ± 1.4 | 6.2 ± 1.2 | 0.101/0.411 | 4-10 × 10⁹/L |
| total count × 10⁹/L | | | | | | |

Three-week administration of probiotic yoghurt did not affect adversely the lipid metabolism parameters (Table 20). Total cholesterol and cholesterol fractions (HDL, LDL-cholesterol and triglycerides) values remained within normal range.

TABLE 20
Blood serum biochemical parameters of healthy volunteers after the
consumption of L. plantarum Tensia DSM 21380 containing yoghurt

| | | | | | P values |
|---|---|---|---|---|---|
| | Probiotic yoghurt | | Control yoghurt | | (BL1 vs BL2, |
| | BL1 | PRO | BL2 | PL | PRO vs PL) |
| Glucose mmol/l | 5.1 ± 0.5 | 5.4 ± 0.5 | 5.3 ± 0.5 | 5.4 ± 0.4 | 0.036/0.144 |
| | | | | | (0.212, 0.757) |
| total | 4.7 ± 0.7 | 4.8 ± 0.7 | 5.0 ± 0.6 | 4.7 ± 0.,6 | 0.315/0.020 |
| cholesterol, | | | | | (0.018, 0.271 |
| mmol/l | | | | | |
| HDL-cholesterol, | 5.0 ± 1.1 | 5.0 ± 1.2 | 5.1 ± 1.4 | 5.0 ± 31.0 | 0.808/0.411 |
| mmol/l | | | | | (0.676, 0.411) |
| LDL-cholesterol, | 1.7 ± 0.3 | 1.6 ± 0.4 | 1.6 ± 0.4 | 1.6 ± 0.4 | 0.158/0.432 |
| mmol/l | | | | | (0.412, 0.747) |
| triglycerides, | 3.1 ± 1.1 | 3.2 ± 1.0 | 3.2 ± 1.2 | 3.1 ± 1.0 | 0.396/0.230 |
| mmol/l | | | | | (0.449; 0.170) |

BL1—at the beginning of the trial, PRO—after probiotic cheese consumption, BL 2—recovery

After administration of the probiotic yogurt slightly increased blood serum glucose values were observed (from 5.1 to 5.4 p value=0036), which, however, remained within the normal range (3.3 to 5.5 mmol/1). Thus, it can be argued, that the yoghurt containing Lactobacillus plantarum Tensia DSM 21380 does not cause systemic inflammation, total allergic sensitisation, or impair the work of essential organs.

### Example 7

### Production of Dietary Supplement Comprising Lactobacillus plantarum Tensia DSM 21380

Firstly, L. plantarum Tensia DSM 21380 freeze-dried (lyophilized) culture was produced as follows. The strain L. plantarum Tensia DSM 21380 inoculated from the stock culture into the bioreactor Bioflo 415 (New Brunswick Scientific) and cultivated at 37° C. for 24 h in MRS medium. The pH of the medium was kept at 6.25. When the bacterial culture reached the maximum mass density, the contents of the reactor was cooled down to temperature 12° C. and pumped into the high-speed laboratory centrifuge Z 41 (Carl Padberg Zentrifugenbau GmbH). The biomass of L. plantarum Tensia DSM 21380 was separated from medium at 20 000 rpm and removed from the centrifuge cylinder aseptically.

Secondly, the biomass of L. plantarum Tensia DSM 21380 was mixed with suitable cryoprotectant (e.g. 2%-10% (w/v) of skim milk powder, buttermilk powder or whey powder and 2%-20% (w/v) of saccharose or sorbite) and said mixture was lyophilised (freeze-drier Christ Delta 2-24 LSC, John Morris Scientific) during 24 to 48 h.

The freeze-dried bacterial culture (L. plantarum Tensia DSM 21380 with cryoprotectant) was removed aseptically from the pans and grinded into powder. Lyophilized bacterial culture was packed in clean, closed containers and stored at −18° C. to −25° C.

For production of dietary supplement, in order to obtain the desired viable cell count, food-grade lactose or microcrystalline cellulose was added as bulking agent.

The germ count of the strain Lactobacillus plantarum Tensia DSM 21380 in the centrifuged biomass was from 5×10¹⁰ to 5×10¹³ CFU/g.

Viable cell count of L. plantarum Tensia DSM 21380 in freeze-dried culture (i.e. mix of biomass of L. plantarum Tensia with cyroprotectant) was from 1×10¹¹ to 5×10¹² CFU/g.

For the preparation of a dietary supplement with defined viable cell counts of Lactobacillus plantarum Tensia DSM 21380, food-grade lactose or microcrystalline cellulose as a bulking agent was added to the Lactobacillus plantarum Tensia DSM 21380 freeze-dried culture with viable cell count from 1×10¹¹ to 5×10¹² CFU/g.

In order to get viable cell count 1×10⁸ CFU/g of Lactobacillus plantarum Tensia DSM 21380 in the final mix of Lactobacillus plantarum Tensia DSM 21380 and bulking agent, 0.002%-0.1% of Lactobacillus plantarum Tensia DSM 21380 (viable cell count density 1×10¹¹ to 5×10¹² CFU/g in freeze-dried culture) was added to the bulking agent.

In order to get viable cell count 1×10¹¹ CFU/g of Lactobacillus plantarum Tensia DSM 21380 in the final mix of Lactobacillus plantarum Tensia DSM 21380 and bulking agent, 0.2%-100% of Lactobacillus plantarum Tensia DSM 21380 (viable cell count density 1×10¹¹ to 5×10¹² CFU/g in freeze-dried culture) was added to the bulking agent.

### Example 8

### Effect of a Dietary Supplement Containing the Probiotic Strain of Lactobacillus plantarum Tensia DSM 21380 on Blood Indices and Intestinal Microflora of Healthy Volunteers

A randomized placebo-controlled parallel trial (international registration number of the clinical trial ISRCTN24502121) was carried out to assess the safety of the consumption of a dietary supplement with Lactobacillus plantarum Tensia DSM 21380. The trial was carried out on 35 volunteers of both sexes consuming either probiotic dietary supplement (daily dose of Tensia 10¹⁰ CFU) or placebo for 4 weeks.

The consumption of probiotic dietary supplement containing L. plantarum strain Tensia was well tolerated by volunteers as no adverse gastrointestinal effects (i.e. no abdominal discomfort like abdominal pain, flatulence or bloating, no negative shifts in values of systemic inflammation markers, no allergic sensibilisation, no harm to essential organs nor unwanted changes in the glucose content in blood serum or in lipid metabolism) were found during the trial. The administrated strain L. plantarum Tensia DSM 21380 was detectable in study participants with RAPD-PCR in faecal samples after the probiotic treatment in the range of 4.0-6.0×10¹⁰ CFU.

Thus, the appearance of the Lactobacillus plantarum Tensia DSM 21380 in faeces of volunteers after administration with dietary supplement in daily dose of 10¹⁰ CFU proves that the probiotic strain Tensia DSM 21380 withstands the transit through upper parts of gastrointestinal tract (i.e. stomach). Appearance of the strain Tensia DSM 21380 in faeces serves as a surrogate marker for the proof that the L. plantarum Tensia DSM 21380 reaches the target site in human body i.e. into small intestine.

What is claimed is:

1. An antimicrobial and antihypertensive probiotic composition, comprising probiotic microorganism strain Lactobacillus plantarum Tensia DSM 21380.

2. The composition of claim 1, wherein said composition is selected from the group consisting a pharmaceutical composition, a food composition and a dietary supplement.

3. A food product comprising the probiotic microorganism strain Lactobacillus plantarum Tensia DSM 21380.

4. The food product of claim 3, wherein the food product is a dairy product.

5. The food product of claim 4, wherein the food product is a fermented milk product.

6. The food product of claim 5, wherein the food product is cheese.

7. A method for suppressing contaminating microbes in a food product comprising a step of adding to a food product the probiotic microorganism Lactobacillus plantarum Tensia DSM 21380.

8. The method of claim 7, wherein said microbes are at least one of the group consisting of a non-starter lactobacilli, pathogens originated from raw milk, and a pathogen occasionally contaminating a food product after preparation.

9. The method of claim 7, wherein the food product is a dairy product.

10. The method of claim 7, wherein the dairy product is a fermented milk product, cheese, cottage cheese, curds, yoghurt, ice-cream, butter, or spread cheese.

11. The method for the extension of the shelf life of a food product comprising a step of adding the probiotic microorganism Lactobacillus plantarum Tensia DSM 21380 to a food product.

12. A dietary supplement, comprising the probiotic microorganism strain Lactobacillus plantarum Tensia DSM 21380.


SEQUENCE LISTING (10 sequences)

SEQ ID NO: 1 - DNA, 17 bp, Lactobacillus plantarum
  primer_bind (375855) (375871): Designation of the sequence ElF, location the sequence measured against the positive control Lactobacillus plantarum WCFSl
atcggcaggcccaacag

SEQ ID NO: 2 - DNA, 21 bp, Lactobacillus plantarum
  primer_bind (375954) (375934): Designation of the sequence ElR, location the sequence measured against the positive control Lactobacillus plantarum WCFSl
gcgtgaccgtgaattaaatgc

SEQ ID NO: 3 - DNA, 21 bp, Lactobacillus plantarum
  primer_bind (375837) (375857): Designation of the sequence F1F, location of the sequence measured against the positive control Lactobacillus plantarum WCFS1
gacagcgctaatgacccaatc

SEQ ID NO: 4 - DNA, 21 bp, Lactobacillus plantarum
  primer_bind (375954) (375934): Designation of the sequence F1R, location the sequence measured against the positive control Lactobacillus plantarum WCFS1
gcgtgaccgtgaattaaatgc

SEQ ID NO: 5 - DNA, 19 bp, Lactobacillus plantarum
  primer_bind (366953) (366971): Designation of the sequence J1F, location the sequence measured against the positive control Lactobacillus plantarum WCFS1
cccaacaaccccgttcaac

SEQ ID NO: 6 - DNA, 26 bp, Lactobacillus plantarum
  primer_bind (367060) (367035): Designation of the sequence J1R, location of the sequence measured against the positive control Lactobacillus plantarum WCFS1
tgctacagtaaagggagaggtatgac

SEQ ID NO: 7 - DNA, 27 bp, Lactobacillus plantarum
  primer_bind (366670) (366696): Designation of the sequence K2F, location the sequence measured against the positive control Lactobacillus plantarum WCFS1
acttattataatcccttgaaccaccaa

SEQ ID NO: 8 - DNA, 25 bp, Lactobacillus plantarum
  primer_bind (366796) (366772): Designation of the sequence K2R, location the sequence measured against the positive control Lactobacillus plantarum WCFS1
gctgacgctgaaaagaatatttctg

SEQ ID NO: 9 - DNA, 21 bp, Escherichia coli
  primer_bind (503858) (503872): Designation of the sequence 968-GC-f, location the sequence measured against the positive control Lactobacillus plantarum WCFS1
gggaacgcgaagaaccttagc

SEQ ID NO: 10 - DNA, 17 bp, Escherichia coli,
  primer_bind (504289) (504273): Designation of the sequence 1401-r location the sequence measured against the positive control Lactobacillus plantarum WCFS1
cggtgtgtacaagaccc